(19) United States
(12) Patent Application Publication
(10) Pub. No.: US 2026/0157998 A1
(43) Pub. Date: Jun. 11, 2026

(54) METHODS AND COMPOSITIONS FOR TREATING NEURODEGENERATIVE DISEASES

(71) Applicant: University of Rhode Island Board of Trustees, Kingston, RI (US)

(72) Inventors: Claudia Fallini, North Kingstown, RI (US); Michelle Gregoire, Goshen, CT (US)

(21) Appl. No.: 19/394,575

(22) Filed: Nov. 19, 2025

Related U.S. Application Data
(60) Provisional application No. 63/722,070, filed on Nov. 19, 2024.

(51) Int. Cl. A61K 31/4015; A61P 25/28; C12Q 1/48

(52) U.S. Cl. CPC A61K 31/4015; A61P 25/28; C12Q 1/485; G01N 2333/912; G01N 2800/52

(57) ABSTRACT

This disclosure provides a method of treating a neurodegenerative disease that includes administering to a subject in need thereof an effective amount of an agent that specifically reduces a level or activity of protein kinase A regulatory (PKA) subunit 1β (R1β).

### RELATED APPLICATION

This application claims the benefit of U.S. Provisional Application No. 63/722,070, filed on Nov. 19, 2024, the entire content of which is hereby incorporated by reference herein.

### GOVERNMENT SUPPORT

This invention was made with government support under R01 NS116143 awarded by National Institute of Health. The government has certain rights in the invention.

### SEQUENCE LISTING

A computer readable file containing a sequence listing is being electronically co-filed herewith via Patent Center. The computer readable file, submitted under 37 CFR § 1.831 (e), will also serve as the copy required by 37 § CFR 1.831 (c). The file (filename “UNRI-003-101 SEQ LIST.XML”) was created on Nov. 18, 2025, and has a size of 8,823 bytes. The content of the computer readable file is hereby incorporated by reference in its entirety.

### BACKGROUND OF THE INVENTION

Synaptic loss and dendritic degeneration are common pathologies in several neurodegenerative diseases characterized by progressive cognitive and/or motor decline, such as Alzheimer's disease (AD) and frontotemporal dementia/amyotrophic lateral sclerosis (FTD/ALS). An essential regulator of neuronal health, the cAMP-response element binding protein (CREB) positively regulates synaptic growth, learning, and memory. Phosphorylation of CREB by protein kinase A (PKA) and other cellular kinases promotes neuronal survival and maturation via transcriptional activation of a wide range of downstream target genes. CREB pathway dysfunction has been strongly implicated in AD pathogenesis, and recent data suggests that impaired CREB activation may contribute to disease phenotypes in FTD/ALS as well. However, the mechanisms behind reduced CREB activity in FTD/ALS pathology are not clear. Currently there are no disease-changing treatments available for FTD/ALS. Antidepressants can help alleviate psychiatric symptoms, and the few drugs approved for these diseases can only extend life expectancy by a few months.

There is a need to elucidate the mechanism behind early CREB pathway dysfunction and discern a feasible therapeutic target for the treatment of FTD/ALS and other neurodegenerative diseases.

### SUMMARY OF THE INVENTION

In one aspect, this disclosure provides a method of treating a neurodegenerative disease, comprising: administering to a subject in need thereof an effective amount of an agent that specifically reduces a level or activity of protein kinase A regulatory (PKA) subunit 1β (R1β).

In some embodiments of the method, the agent specifically reduces a level of R1β. In some embodiments, the agent is an antisense oligonucleotide (e.g., a morpholino oligonucleotide), a small interfering RNA (siRNA), a microRNA (miRNA), a short hairpin RNA (shRNA), a ribozyme, a proteolysis targeting chimera (PROTAC), an activator of endogenous proteasomal-mediated degradation of R1β, or a combination thereof.

In some embodiments, the agent used in the method inhibits an activity of R1β. In some embodiment, the activity is binding to a PKA catalytic subunit or binding to an A-kinase anchoring protein. In one embodiment, the agent is an antibody, small molecule, peptide specific for R1β, or a combination thereof.

In one embodiment of the method of treating a neurodegenerative disease, the subject has an increased level or activity of R1β as compared to a subject without a neurodegenerative disease. In some embodiments, the method further includes determining the activity or level of R1β in the subject before, during, and/or after administration of the agent.

In some embodiments, the subject in need of treatment has one or more mutations associated with a neurodegenerative disease, such as a mutation in the C9ORF72 or SOD1 gene.

In some embodiments, the neurodegenerative disease treated by the method is Parkinson's disease (PD), Alzheimer's disease (AD), amyotrophic lateral sclerosis (ALS), frontotemporal dementia (FTD), multiple sclerosis (MS), progressive supranuclear palsy (PSP), Lewy body dementia (LBD), chronic traumatic encephalopathy (CTE), spinocerebellar disease, Huntington's disease, Fragile X-associated tremor/ataxia syndrome (FXTAS), or Limbic predominant age-related TDP-43 encephalopathy (LATE).

In some embodiments, the treatment method includes administering to the subject the agent that specifically reduces a level or activity of R1β and one or more other agents for treating the neurodegenerative disease.

In other aspects, the present disclosure provides a composition for treating a neurodegenerative disease that includes an agent that specifically reduces a level or activity of R1β. Also contemplated herein is a method of identifying a candidate drug for treating a neurodegenerative disease, including assaying or determining the ability of a test compound to modulate a level or activity of R1β in an in vitro, ex vivo, or in vivo system.

### BRIEF DESCRIPTION OF THE DRAWINGS

FIG. 1 shows I³ (integrated, inducible, isogenic) neuronal differentiation and characterization. A. Schematics of the i³ differentiation timeline and steps used in this study; B. The efficiency of differentiation was quantified as percentage of MAP2-positive over all DAPI-positive cells. For either iPSC line, no difference between ISO and HRE neurons was observed (paired t test, n=5 biological replicates, ns: not significant); C. Representative quantification of two-week-old ISO and HRE neurons showing similar expression level of the glutamate AMPA receptor subunit GluA1 (paired t test, n=3 biological replicates [2 CS52 and 1 24a]; ns: not significant); D. Quantification of neuronal survival at day 14 show no difference between HRE and ISO cells from both 24a and CS52 lines (paired t test, n=4 biological replicates; ns: not significant). Bars are mean and SEM, symbols represent independent biological replicates. Triangles identify 24a lines, while circles represent CS52 neurons.

FIG. 2 shows that HRE cortical neurons display a reduction in dendritic branching and synaptic protein expression. A-B. Sholl analysis demonstrated a reduced number of dendritic crosses per concentric shells across the cell radius (5 μm increments) in HRE neurons compared to ISO controls (A). Statistical analysis of the area under the curves (B), calculated by plotting the number of crossing intersections across the radius of the cell, revealed a significant difference (paired t test, n=4 biological replicates, *p<0.05). C-D. Skeleton analysis of HRE compared to ISO neurons revealed a significantly lower number of junctions (C) and branches (D). The frequency distribution of branch and junction numbers is shown on the graphs on the right in C and D (Fisher's exact test, n=36 for both HRE and ISO from 4 biological replicates, *p<0.05). E. Quantification of maximum branch length in ISO and HRE neurons. Data are shown as scattered dot plots of individual cells (left; unpaired t test, n=36 ISO and HRE, *p<0.05) or as the average for each biological replicate (right; paired t test, n=4 biological replicates, *p<0.05). F. Quantification of dendritic surface expression of GluN2A (green) revealed significantly lower levels in dendrites of HRE i³CNs compared to ISO controls. Data are shown as scattered dot plots of individual cells (left; Mann Whitney test, n=49 ISO and 52 HRE neurons, ***p<0.001) or as the average for each biological replicate (right; paired t test, n=6 biological replicates, *p<0.05). G-H. Representative western blot (G) and quantification (H) of PSD95 levels relative to GAPDH showed a significant reduction in in HRE i³CNs compared to ISO controls (paired t test, n=6, **p<0.01). Bars and lines are mean and SEM in all but D and E, where they indicate the median. In all, triangles and circles indicate data obtained from 24a and CS52 neurons, respectively.

FIG. 3 shows characterization of dendritic and synaptic defects in 24a and CS52 i³CNs. A-F. Quantification and statistical analysis as shown in FIG. 2 of number of junctions (A, D), branches (B, E) and dendritic levels of GluN2A (C, F) performed separately on HRE and ISO neurons differentiated from 24a (A-C) or CS52 (D-F) iPSCs. Similar trends for all markers are evident in both lines. Graph bars in A-E represent frequency distribution for each bin value shown on the X axis (n=20 ISO and 19 HRE in A-B from 2 biological replicates and n=16 ISO and 17 HRE in D-E from 2 biological replicates; Fisher's exact test, **p<0.01, n.s. not significant; n=26 ISO and 28 HRE in C and n=23 ISO and 24 HRE in F from 3 independent replicates each, Mann Whitney test, *p<0.05, **p<0.01). G-H. Representative western blot (G) and quantification (H) of PSD95 levels relative to GAPDH showed a significant reduction in in HRE i³CNs compared to ISO controls (paired t test, n=6, **p<0.01).

FIG. 4 shows that pCREB levels are significantly lower in C9 HRE neurons. A. Quantification of the mean fluorescence intensity (mFI) demonstrates a significant reduction in pCREB levels in HRE neurons relative to ISO controls. Data are shown as scattered dot plots of individual cells (left; unpaired t test, n=88 ISO and 93 HRE from 6 independent replicates, **p<0.01) or as the average for each biological replicate (right; paired t test, n=6 biological replicates, *p<0.05). B-E. Representative western blot (B) showing pCREB and CREB expression compared to GAPDH, used as a loading control. Quantification of band intensities revealed that CREB levels are not significantly different between ISO and HRE neurons (C), but that levels of pCREB (D) as well as the ratio of pCREB to CREB (pCREB/CREB, E) are significantly lower in mutant i³CNs compared to controls (paired t test, n=4 biological replicates, *p<0.05, **p<0.01, ns: not significant). F. qPCR analysis of BDNF mRNA levels reveals a significant decrease in C9 HRE neurons (paired t test, n=7 biological replicates, *p<0.05). For all, data from 24a neurons are shown as triangles while CS52 are represented by circles. Lines are mean and SEM.

FIG. 5 shows quantification of pCREB levels in 24a and CS52 i³CNs. Quantification and statistical analysis as shown in FIG. 4 of pCREB levels in 24a (A) and CS52 (B) neurons. Data are displayed as scattered dot plots of individual cell values. Lines represent mean and SEM (n=55 ISO and 58 HRE from 4 replicates in A, n=33 ISO and 35 HRE from 2 replicates in B, unpaired t test, *p<0.05).

FIG. 6 shows CREB activation in C9 HRE iPSCs and early neurons. A-C. The cellular levels of pCREB and CREB were analyzed in iPSCs by western blot. While we found a small increase in CREB levels in HRE cells (A), no significant difference in pCREB (B), or pCREB/CREB ratio (C) were detected (paired t test, n=5 biological replicates, ns: not significant). D-E. Immunofluorescence analysis of CREB (quantified in D) and pCREB (quantified in E) confirms no changes in HRE or ISO iPSCs (unpaired t test, n=16 ISO and 17 HRE colonies from 3 biological replicates). F-G. Quantification of mean fluorescence intensities revealed significantly lower levels for both CREB (F) and pCREB (G) in HRE neurons, indicating that CREB pathway dysfunction co-occurs alongside neuronal maturation (unpaired t test, n=84 ISO and HRE cells from 2 biological replicates, **p<0.01, ****p<0.0001). Bars and lines are mean and SEM.

FIG. 7 shows that KCl-dependent activation of CREB is maintained in HRE neurons. A. Schematics of the experimental timeline. 13CNs were treated with 1 μM tetrodotoxin (TTX) for 8 hours followed by 55 mM KCl or vehicle alone (Untr.) for 30 or 60 minutes. B-C. Quantification of pCREB levels in ISO and HRE neurons treated with KCl for 30 minutes (B) or 1 hour (C). No significant difference in pCREB increase was observed. Data are shown as scattered dot plots (top graphs in B and C) or as the average for each biological replicate (bottom graphs in B and C), with triangles indicating 24a and circles CS52 neurons. Statistical analyses were performed via two-way ANOVA with Šídák's post hoc test (top graphs in B and C), and with RM two-way ANOVA with Fisher's LSD (bottom graphs in B and C; n=56-35 ISO and 56-52 HRE from 3 biological replicates in B, n=85-88 ISO and 98-100 HRE from 7 biological replicates in C; *p<0.05, ****p<0.0001, ns: not significant).

FIG. 8 shows that PKA-dependent activation of CREB is selectively impaired in HRE cortical neurons. Quantification of mean fluoresce intensity (mFI) of pCREB (A) and of PKA catalytic subunit (Ca) in the whole cell (B) or nucleus (C) in ISO and HRE neurons before and after stimulation with FSK. D. Representative western blot of pCREB in HRE and ISO neurons treated with 5 μM FSK for 30 minutes. GAPDH was used as leading control. E-F. Quantification of pCREB band intensities (C) or fold change of pCREB levels in FSK-treated neurons over TTX-only (Untr.) (D) in HRE and ISO neurons. Data are shown as scattered dot plots (n=102-100-101 ISO and 90-100-104 HRE, left graphs in A-C, n=4 in G) or as the average of the FSK/Untr fold change for each biological replicate (n=6, right graphs in A-C, n=4 in H). Statistical analyses were performed via two-way ANOVAs with Šídák's post hoc test in A, B, C and E (left graphs), RM two-way ANOVA with Fisher's LSD post hoc test in A, B, and C (right graphs) and E, and with paired t test in F (*p<0.05, **p<0.01, ***p<0.001, ****p<0.0001, ns: not significant). For all, bars or lines indicate mean and SEM, triangles and circles identify 24a and CS52 datapoints, respectively

FIG. 9 shows that FSK treatment increases CAMP levels and active PKA enzyme. A. Schematic representation of the pathway involved in PKA activation. The adenylyl cyclase (AC) is artificially activated FSK to increase the conversion of cAMP from ATP. Four CAMP molecules bind to the regulatory (R) dimer of PKA, leading to the release of the catalytic (C) active subunits. Phosphodiasterases (PDE) convert CAMP in the inactive AMP molecule. B. Quantification of cAMP cellular levels in ISO and HRE neurons before and after FSK stimulation (two-way ANOVA with Šídák's post hoc test, n=2). C. Quantification of PKA Ca levels HEK293 cells before and after 10 minutes FSK treatment. To assess that the change in mFI was dependent on increased antibody access of the subunit and not due to increased protein expression, cells were pre-treated with 10 μg/ml cycloheximide for 1 hour to block protein synthesis. An increase in Ca levels was observed in both conditions following FSK stimulation (C, D) suggesting the antibody used is relatively specific for the active PKA enzyme. Data are shown as scattered dot plots (C) or as the average for each biological replicate (D). Statistical analyses were performed via either two-way ANOVA with Šídák's post hoc test (C) or RM two-way ANOVA with Fisher's LSD test (D) (n=76, 72, 75, and 75 from 3 biological replicates as they appear on the graphs; *p<0.05, ****p<0.0001, ns: not significant).

FIG. 10 shows that CAMP-dependent activation of PKA and CREB is similarly diminished in both 24a and CS52 HRE neurons. A-B. Analysis of pCREB mean fluorescence intensity (mFI) as in FIG. 8 in 24a (A) and CS52 (B) ISO and HRE neurons treated with FSK (n=74-47 for Untr., 72-68 for 30′ FSK, 72-78 for 60′ FSK from 3 biological replicate in A; n=45-36 for Untr., 42-43 for 30′ FSK, 43-37 for 60′ FSK from 2 biological replicate in B). C-D. Analysis of total PKA mFI in 24a (C) and CS52 (D) ISO and HRE neurons treated with FSK (n=38-40 for Untr., 40-39 for 30′ FSK, 43-45 for 60′ FSK from 3 biological replicate in C; n=35-31 for Untr., 30-37 for 30′ FSK, 34-30 for 60′ FSK from 2 biological replicate in D). E-F. Analysis of nuclear PKA mFI in 24a (E) and CS52 (F) ISO and HRE neurons treated with FSK (n=38-39 for Untr., 40-37 for 30′ FSK, 43-45 for 60′ FSK from 3 biological replicate in E; n=35-31 for Untr., 30-37 for 30′ FSK, 34-30 for 60′ FSK from 2 biological replicate in E). For all, data are shown as scattered dot plots (left) or as the average of the FSK/Untr fold change for each biological replicate (right). Statistical analyses were performed via two-way ANOVAs with Tukey's post hoc test and for fold change graphs, with RM two-way ANOVA and Fisher's LSD test (*p<0.05, **p<0.01, ****p<0.001).

FIG. 11 shows that PKA inhibition via H-89 treatment leads to a decrease in pCREB levels and a decrease in dendritic arborization. A. Quantification of pCREB levels in ISO neurons treated chronically with 10 μM H-89 or vehicle alone (Untr.) for 14 days. Data are shown as scattered dot plots (n=35 Untr, and 38 H-89 treated ISO neurons, unpaired t test, ****p<0.0001) or as the average for each biological replicate (n=3, paired t test, *p<0.05). B-C. Quantification of pCREB levels by western blot confirmed the IF data. A representative blot is shown in B (paired t test, n=5, *p<0.05). D-E. Sholl analysis (D) and quantification of the area under the curve (E) demonstrated a reduced number of dendritic crosses across the cell radius in H-89 treated neurons (paired t test, n=3 biological replicates, **p<0.001). F-G. Skeleton analysis of neuronal morphology shows a significantly lower number of junctions (F) and branches (G) in H-89 treated neurons, suggesting a greatly reduced dendritic complexity. The frequency distribution of the number of branches and junctions is shown on the graphs on the right in F and G (Fisher's exact test, n=41 Untr, and 50 H-89 ISO neurons from 3 biological replicates, ****p<0.0001). H. Quantification of maximum branch length in ISO neurons treated with H-89 versus untreated controls. Data are shown as scattered dot plots of individual cells (left; unpaired t test, n=41 Untr, and 50 H-89 ISO neurons, **p<0.01) or as the average for each biological replicate (right; paired t test, n=3 biological replicates, p=0.18). Bars and lines represent mean and SEM for all but F and G, where lines indicate the median. Triangles and circles identify 24a and CS52 datapoints, respectively

FIG. 12 shows that PKA inhibitor H-89 reduces CREB activity and dendritic branching similarly in both 24a and CS52 neurons. A-B. Representative western blot (A) and quantification of pCREB levels (B) in ISO neurons treated with H-89 or vehicle alone (Untr.) (paired t test, n=5, *p<0.05). C-D. Scattered dot plots of the number of junctions (C) and dendrites (D) in ISO neurons treated with H-89 compared to controls, as shown in FIG. 4 (n=41 Untr, and 50 H-89 ISO neurons from 3 biological replicates). E-L. Quantification and statistical analysis as shown in FIG. 11 of number of junctions (E, I), branches (F, J), max branch length (G, K), and pCREB mFI (H, L) performed separately on untreated or H-89-treated ISO neurons differentiated from 24a (E-H) or CS52 (I-L) iPSCs. Similar trends for all markers are evident in both lines. Graph bars in E-F and I-J represent frequency distribution for each bin value shown on the X axis (n=27 Untr and 25 H-89 in E-G from 2 biological replicates and n=16 Untr and 19 H-89 in I-K from 1 biological replicate; Fisher's exact test; n=22 Untr and 26 H-89 in H from 2 biological replicates and 13 Untr and 18 H-89 in L from 1 biological replicate in H, unpaired t test, *p<0.05, ***p<0.001, **p<0.0001, n.s. not significant).

FIG. 13 shows that elevated levels of PKA regulatory subunits contribute to decreased pCREB levels in HRE neurons. A-B. Quantification of total (A) or nuclear (B) Ca mFI revealed a significant reduction in HRE neurons compared to ISO controls. Data are shown as scattered dot plots (left graphs, unpaired t test, n=78 ISO and 77 HRE neurons, ****p<0.0001) or as the average for each biological replicate (right graphs, paired t test, n=5, *p<0.05). C-E. Representative blots (C) and quantification of Ca (D), R1β (E) and the ratio of R1β to Cα (F) reveal an imbalance in the levels of the catalytic versus the regulatory subunits in HRE neurons (paired t test, n=5, *p<0.05, ns: not significant). G. Quantification of pCREB levels in control ISO neurons transduced with lentiviral vectors expressing GFP and either mCherry or the untagged R1β subunit. Overexpression of R1β is sufficient to reduce endogenous levels of pCREB. Data are shown as scattered dot plots (left graph, unpaired t test, n=28 mCherry and 34 R1β, *p<0.05) or as the average for each biological replicate (right graph, paired t test, n=3, *p<0.05). For all, lines represent mean and SEM. Triangles and circles identify 24a and CS52 datapoints, respectively.

FIG. 14 shows that PKA C/R subunit balance is altered in 24a and CS52 HRE neurons driving pCREB defects. Quantification of total (left graphs) or nuclear (right graphs) PKA Ca mFI in ISO and HRE neurons differentiated from 24a (A) or CS52 (B) iPSCs, as shown in FIG. 13. A significant reduction was observed in both lines (unpaired t test, n=40 ISO and 46 HRE in A, n=38 ISO and 21 HRE in B, *p<0.05, ****p<0.0001). C. Quantification of pCREB levels in control ISO neurons transduced with lentiviral vectors expressing GFP and either mCherry or the untagged R1β subunit. Data are shown as scattered dot plots (top graph, unpaired t test, n=28 mCherry and 34 R1β, *p<0.05) or as the average for each biological replicate (bottom graph, paired t test, n=3, *p<0.05). For all, lines represent mean and SEM.

FIG. 15 shows that PKA dysregulation and pCREB reduction are conserved in C9-ALS postmortem brain tissue. A-C. Quantification of PKA Ca cytoplasmic (A) and nuclear levels (B) shows a significant reduction in C9-ALS patients versus control. A similar downregulation in the nuclear levels of pCREB (C) was also observed (unpaired t test, n=5 C9-ALS patients and 5 controls, *p<0.05, **p<0.01). D. Quantification of R1β levels show significantly higher levels in C9-ALS patients compared to controls (unpaired t test, n=5 C9-ALS patients and 5 controls, **p<0.01). For all, bars indicate mean and SEM.

FIG. 16 shows immunohistochemistry analysis of C9-ALS and control tissues. A. While pCREB and PKA Ca are both reduced in C9-ALS neurons (see FIG. 15), MAP2 levels are unchanged, and no correlation between Ca and MAP2 levels can be discerned, which confirms that the differences observed are biologically relevant and not due to poor quality of the tissues or staining protocol. B-D. Quantification of Nuclear PKA Cα (B), pCREB (C) and PKA R1β (D) levels in each individual control and C9-ALS patient. The dashed line represents the average of all controls

FIG. 17 shows that PDE 4 is expressed in i³CNs and is targeted by rolipram treatment. A. Schematics of Rolipram mechanism of action. B. Q-PCR quantification of the expression level of the four main PDE4 isoform expressed in neurons (two-way ANOVA with Tukey's post hoc test, n=3, **p<0.01). C-E. Quantification of the change in expression PDE 4A, 4B, and 4D following rolipram treatment (two-way ANOVA with Tukey's post hoc test, n=3, not significant). F. Quantification of cellular cAMP levels in ISO and HRE neurons after chronic rolipram treatment (two-way ANOVA with Šídák post hoc test, n=3, *p<0.05).

FIG. 18 shows that Rolipram treatment rescues PKA subunit homeostasis and pCREB levels in HRE neurons. A-C. Quantification of Ca (A) and R1β (B) band intensities shows a small but not significant effect in both ISO and HRE neurons, which leads to a significant decrease in the R/C ratio in HRE neurons (C; RM two-way ANOVA with Fisher's LSD post hoc test, n=6 biological replicates, *p<0.05, ns: not significant). D. Quantification of ROL effects on pCREB levels in ISO and HRE neurons. A significant increase in pCREB was observed in HRE neurons treated with ROL for two weeks. Data are shown as scattered dot plot (two-way ANOVA with Fisher's LSD post hoc test, n=45-54 ISO cells and 43-58 HRE neurons, *p<0.05, ****p<0.0001, ns: not significant) or as average per biological replicate (RM two-way ANOVA with Fisher's LSD post hoc test, n=3, *p<0.05, ns: not significant). E. Quantification of pCREB levels in HRE neurons treated with ROL for 2 weeks (paired t test, n=5, *p<0.05). Histone H3 (H3) was used as loading control. For all, lines represent mean and SEM. Triangles and circles identify 24a and CS52 datapoints, respectively.

FIG. 19 shows that Rolipram normalizes PKA subunit balance and pCREB levels in both 24a and CS52 neurons. A-B. Quantification of PKA Ca (A) and R1β (B) band intensities (data not shown); RM two-way ANOVA with Fisher's LSD post hoc test, n=6 biological replicates ns: not significant. C-D. Quantification of pCREB as shown in FIG. 18B in 24a (C) and CS52 ISO and HRE neurons (D). Data are shown as scattered dot plots, with lines indicating mean and SEM (two-way ANOVAs with Fisher's LSD post hoc test, n=18-12 ISO and 15-16 HRE in A, n=27-42 ISO and 28-42 HRE in B, *p<0.05, ****p<0.0001, ns: not significant)

FIG. 20 shows that ubiquitinated proteins do not accumulate in HRE neurons. A. Quantification of Ubiquitin-positive protein levels show no significant difference between mutant and isogenic controls (paired t test, n=6 biological replicates, ns: not significant). B-E. Plotting of the ubiquitin band intensity in 24a (B) and CS52 (D) neurons. Quantification of the overall ubiquitin levels in each line shows no significant change in 24a cells (C), while a slight but significant reduction was detected in CS52 HRE neurons (E) (paired t test, ns: not significant, *p<0.05).

FIG. 21 shows that Ibudilast, a non-selective PDE inhibitor, rescues pCREB levels in HRE neurons. Quantification of pCREB levels shows a moderate increase in HRE neurons treated with Ibudilast. Data are shown as scattered dot plot (one-way ANOVA with Tukey's post hoc test, n=65 ISO, 66 HRE Untr and 73 HRE+Ibudilast, ****p<0.0001) or as averages per biological replicate (RM one-way ANOVA with Dunnett's post hoc test, n=4, *p<0.05). Lines are mean and SEM.

FIG. 22 shows that dendritic branching and synaptic proteins are rescued in C9 HRE via CAMP modulation. A-B. Sholl analysis (A) and quantification of the area under each curve (B) of HRE neurons treated with ROL shows a significant increase in dendritic arbor complexity compared to untreated controls (paired t test, n=6 biological replicates, *p<0.05). C-D. Skeleton analysis of MAP2-stained neurons shows that ROL treatment of HRE i³CNs rescues loss of dendrites and dendritic junctions observed in untreated neurons. The frequency distribution of junctions and branch numbers is shown on the graphs on the right (Fisher's exact test, n=95 Untr, and 90 ROL HRE neurons from 6 biological replicates, ****p<0.0001). E. Quantification of maximum branch length in Untr, or ROL-treated HRE neurons. Data are shown as scattered dot plots of individual cells (left; unpaired t test, n=95 Untr, and 90 ROL, ****p<0.0001) or as the average for each biological replicate (right; paired t test, n=6 biological replicates, *p<0.05). F. Quantification of GluN2A surface levels in HRE neurons treated with ROL compared to untreated HRE controls. Data are shown as scattered dot plots of individual cells (left, Mann Whitney test, n=75 Untr, and 68 ROL HRE neurons, ***p<0.001) or as the average for each biological replicate (right, paired t test, n=5). Lines represent mean and SEM in B, E, and F, or the median in C and D. Triangles and circles identify 24a and CS52 datapoints, respectively.

FIG. 23 shows that Rolipram rescues dendritic morphology in 24a and CS52 HRE neurons. A-H. Quantification of the number of branches (A-B), number of junctions (C-D), max branch length (E, G) and GluN2A levels (F, H) in 24a (A, C, E, n=50 Untr and 28 ROL; F n=9 Untr and 11 ROL) or CS52 (B, D, G, n=45 Untr and 52 ROL; H n=66 Untr and 57 ROL) HRE neurons treated with Rolipram compared to untreated controls. In A-D, data are shown as scattered plots or as frequency distribution bar graphs. Statistical analyses were performed as described in FIG. 22. I-J. Scattered dot plot of number of junctions (I) and branches (J) as shown in FIG. 22 D-E. K-L. Representative western blot (K) and quantification (L) of PSD95 levels in HRE neurons treated with ROL or vehicle alone. Histone 3 (H3) was used as a loading control (paired t test, n=6 biological replicates, *p<0.05).

FIG. 24 shows that CREB dysfunction in ALS patients with mutations in the SOD1 gene. A-B. SOD1 mutant neurons in the motor cortex had significantly lower levels of active CREB and PKA compared to control healthy samples. C. Correlation of levels of pCREB and PKA lost in SOD1 ALS samples. D-E. Quantification of nuclear pCREB (D) and PKA Ca (E) levels in each individual control and SOD1 ALS patients.

### DETAILED DESCRIPTION OF THE INVENTION

The data described herein demonstrate that the homeostatic imbalance of the catalytic and regulatory subunits of CREB-activator protein kinase A (PKA) is a main mechanistic driver of CREB dysfunction and that compounds capable of modulating PKA homeostasis significantly rescue CREB-dependent disease pathology in FTD/ALS.

Accordingly, this disclosure contemplates specifically targeting the regulatory subunit of PKA to modulate the homeostatic balance of the catalytic and regulatory subunits for treating neurodegenerative disorders.

### Definition

Protein kinase A (PKA) regulatory subunit 1β or R1β refers to a type I non-catalytic subunit of PKA. Non-limiting exemplary amino acid sequences of R1β and nucleic acid sequences encoding them can be found in public databases. See, e.g., ENSG00000111725; mRNA IDs: MN_006253.5 and XM_005253909.2; and protein ID: NP_006244.2.

As used herein, protein kinase A catalytic (C) subunit, unless otherwise specified, refers to any isoforms of the subunit (e.g., Ca, CB and Cy). In some embodiments, the term refers to the predominant isoform, Ca. Non-limiting exemplary amino acid sequences of the C subunit and nucleic acid sequences encoding them can be found in public databases. See, e.g., PRKACA (ENSG00000072062), PRKACB (ENSG00000142875), and PRKACG (ENSG00000165059)

The term “agent” as used herein means any compound or substance such as, but not limited to, a small molecule, nucleic acid, amino acid, polypeptide, peptide, drug, carbohydrate, aptamer, oligomer of nucleic acids, oligonucleotides, ribozymes, lipoprotein, glycoprotein, antibody, ion, or modifications and combinations thereof. An agent can be any chemical, entity or moiety, including without limitation synthetic and naturally-occurring proteinaceous and non-proteinaceous entities.

The term “modulate” is used consistently with its use in the art, i.e., meaning to cause or facilitate a qualitative or quantitative change, alteration, or modification in a process, pathway, or phenomenon of interest. Without limitation, such change may be an increase, decrease, or change in relative strength or activity of different components or branches of the process, pathway, or phenomenon.

The terms “decrease,” “reduced,” “reduction,” “decrease” or “inhibit” are all used herein generally to mean a decrease by a statistically significant amount. In some embodiments, “reduced,” “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100%, or any decrease between 10-100% as compared to a reference level.

The terms “increased,” “increase” or “enhance” or “activate” are all used herein to generally mean an increase by a statically significant amount. In some embodiments, the terms “increased,” “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, at least about a 3-fold, at least about a 4-fold, at least about a 5-fold, or at least about a 10-fold increase, or any increase between 2-fold and 10-fold or greater as compared to a reference level.

The terms “treat”, “treating”, “treatment”, etc., as applied to an isolated cell, include subjecting the cell to any kind of process or condition or performing any kind of manipulation or procedure on the cell. As applied to a subject, the terms refer to providing medical or surgical attention, care, or management to an individual. As used herein, the term “treatment” includes prophylaxis. The individual is usually ill or injured, or at increased risk of becoming ill relative to an average member of the population and in need of such attention, care, or management. Those in need of treatment include those already diagnosed with a neurodegenerative disorder or showing one or more symptoms of the disorder, as well as those likely to develop a neurodegenerative disorder due to genetic susceptibility, age, or other risk factors. One of skill in the art realizes that a treatment may improve a disease condition but may not be a complete cure for the disease.

As used herein, an “effective amount” of a drug, agent, compound, or pharmaceutical composition is an amount sufficient to produce one or more desired or beneficial results, such as a reduction in at least one symptom of the disease or an improvement in the disease, for example, beneficial or desired clinical results. For purposes of this disclosure, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, prolonging survival, and remission (whether partial or total), whether detectable or undetectable. In some embodiments, desired or beneficial results include (1) a decrease in the level of R1β in the subject as compared to before the treatment, (2) a level of R1β within a “normal” or reference range found in individuals without a neurodegenerative disease, (3) a ratio of R1β level/C subunit level within a “normal” or reference range found in individuals without a neurodegenerative disease, (4) a decreased in the ratio of R1β level/C subunit level as compared to before the treatment, and (5) increased activation of PKA activity or one or more PKA downstream effectors (e.g., CREB phosphorylation) as compared to before the treatment.

As used herein, the term “contacting” is intended to include incubating an agent and a cell together in vitro (e.g., adding the agent to cells in culture). In some embodiments, the term “contacting” is not intended to include the in vivo exposure of cells to the compounds that may occur naturally in a subject (i.e., exposure that may occur as a result of a natural physiological process). In some embodiments, the term is intended to include the in vivo exposure of cells to the compounds.

The terms “subject” and “individual” are used interchangeably herein, refer to an animal. The terms “non-human animals” and “non-human mammals” as used interchangeably herein, includes mammals such as rats, mice, rabbits, sheep, horses, cats, dogs, cows, pigs, and non-human primates. The term “subject” also encompasses any vertebrate including but not limited to mammals, reptiles, amphibians, and fish.

As used in this specification and the appended claims, the singular forms “a,” “an,” and “the” include plural references unless the context clearly dictates otherwise. Thus, for example, references to “the method” includes one or more methods, and/or steps of the type described herein and/or which will become apparent to those persons skilled in the art upon reading this disclosure and so forth.

### Treatment Method

This disclosure is drawn to a method of treating a neurodegenerative disease that specifically targets R1β in order to increase activation of PKA signaling and downstream events such as phosphorylation of CREB.

The treatment method includes a step of administering an affective amount of an agent that specifically reduces a level or an activity of R1β. “Specific” or “preferential” for R1β in the context of an agent means that the agent reacts with, binds to, or associates with an R1β polypeptide or nucleic acid molecule or modulates the level or an activity of R1β more frequently, more rapidly, to a greater extend, with greater duration and/or with greater affinity than it does with alternative polypeptides or nucleic acid molecules. In some embodiments, the agent specifically hybridizes to a nucleic acid molecule encoding an R1β polypeptide or binds to the polypeptide.

An activity of R1β or R1β activity can be any molecular or physiological function of R1β. PKA is a tetramer including two catalytic (C) subunits and two regulatory subunits. The regulatory subunits reversibly bind to the catalytic subunits and prevent the catalytic subunits from phosphorylating target proteins while bound. R1β activity can include, without limitation, binding of R1β to a catalytic subunit, modulation of PKA enzymatic activity, modulation of PKA signaling events, and binding of R1β to A-kinase anchoring proteins. An agent that specifically reduces R1β activity can be an agent that inhibits binding of R1β to a catalytic subunit, increases PKA phosphorylation of targets (e.g., CREB), enhances proteasomal degradation of R1β, or modulates the interaction between R1β and A-kinase anchoring proteins. This can be accomplished, for example, by using an antibody, peptide or another molecule that blocks the catalytic unit binding site on R1β.

The terms “level of R1β”, “R1β level”, and “R1β expression level” etc. are used interchangeably to refer to the level of an R1β protein or a nucleic acid molecule encoding the protein. In other words, an agent or method that specifically reduces R1β level can be an agent or method that decreases the level of an R1β protein or a nucleic acid molecule encoding it. Various techniques for inhibiting the expression of a particular gene are available in the art, including use of an antisense oligonucleotide (e.g., a morpholino oligonucleotide), a small interfering RNA (siRNA), a microRNA (miRNA), a short hairpin RNA (shRNA), and a ribozyme. Given the sequence of the target gene, appropriate inhibitory molecules can be designed. Examples of sequences that can be used in antisense oligonucleotides or shRNAs include GTGTACCCCTGCCCGCGC (SEQ ID NO: 1), GAGCCGCTGTGTGAGGTG (SEQ ID NO: 2), and TCCAAATCAAAGGACAAG (SEQ ID NO: 3). Genome editing techniques such as the CRISPR-CAS9 system can also be utilized to reduce R1β level. R1β level can also be reduced by inducing proteolysis of R1β protein using techniques such as proteolysis targeting chimera (PROTAC). PROTAC is a bifunctional entity including one ligand for binding to a specific protein and another ligand to an E3 ubiquitin (E3) ligase. PROTAC recruits the E3 ligase to the protein and promotes degradation of the protein by the ubiquitin-proteasome system (UPS). For example, a PROTAC for decreasing R1β level can include an E3 ligand, a short linker, and an R1β ligand such as a polypeptide containing the binding site on the PKA catalytic subunit for R1β.

In some embodiments, the method further includes administering another agent for treating a neurodegenerative disease. The other agents include phosphodiesterase (PDE) inhibitors such as rolipram, ibudilast, Roflumilast, Cilomilast, Crisaborole, Roflumilast, and Apremilast, and other drugs such as amantadine, apomorphine, baclofen, carbidopa, carbidopa/levodopa, dantrolene, donepezil, entacapone, galantamine, levodopa, memantine, pramipexole, rasagiline, riluzole, rivastigmine, ropinirole, selegiline, tacrine, tizanidine, and tolcapone. The agent specific for R1β and the other agent can be administered at the same time as a single composition or separate compositions or administered serially (e.g., one before the other).

The agent specific for R1β can be administered to a subject via any suitable routes of administration, e.g., intravenous, intramuscular, intraarterial, intrathecal, intracerebroventricular, intraventricular, intracapsular, intraorbital, intracardiac, intradermal, intranasal, intraperitoneal, intraparenchymal, transtracheal, subcutaneous, subcuticular, intraarticular, subpial, subarachnoid, intraspinal, intracerebral, oral, topical, and intrasternal routes.

Neurodegenerative diseases that can be treated with the method described herein include but are not limited to Parkinson's disease (PD), Alzheimer's disease (AD), amyotrophic lateral sclerosis (ALS), frontotemporal dementia (FTD), multiple sclerosis (MS), progressive supranuclear palsy (PSP), Lewy body dementia (LBD), chronic traumatic encephalopathy (CTE), spinocerebellar disease, Huntington's disease, Fragile X-associated tremor/ataxia syndrome (FXTAS), and Limbic predominant age-related TDP-43 encephalopathy (LATE).

In some embodiments, the method can be used to treat neurodegenerative diseases associated with one or more mutations, such as mutations in the C9ORF72 gene, the SOD1 gene, the ARDBP gene, and/or the FUS gene. A mutation in the C9ORF72 gene can be the expansion of the GGGGCC hexanucleotide sequence in Intron 1 of the C9ORF72 gene. There are more than 100 mutations in the SOD1 gene linked to ALS: SOD1 A4V being the most common in the US, SOD1 I113T being the most common in Europe, and SOD1 H46R being the most common in Asia. See Ruffo et al., Genes 2022, 13 (3): 537. In some embodiments, the method described herein can be used to treat neurodegenerative diseases associated with defective CREB pathway activation such as lower level of active phosphorylated CREB, increased R1β level, lower PKA C subunit level, or higher R1β/C subunit ratio.

In any of the methods described herein, the level of R1β can be determined in the subject at one or more time points before, during and/or after the treatment. Determining the level in a subject can evaluate whether the subject is suitable for the treatment or whether the treatment is effective. A subject exhibiting an elevated R1β level or R1β/C subunit ratio may be particularly suited for the treatment. A decreased R1β level or R1β/C subunit ratio after the treatment is administered suggests that the treatment is effective. The R1β level can be assayed at either the mRNA level or at the protein level. Methods of measuring mRNA levels and protein levels in a sample are well known in the art, e.g., Western blotting, ELIZA, PCR, and quantitative PCR.

### Pharmaceutical Compositions

The agent specific for R1β can be formulated as a pharmaceutical composition containing one or more pharmaceutically acceptable carriers. The composition can be formulated to facilitate crossing the blood brain barrier (BBB), such as encapsulating the agent in nanoparticles, e.g., lipid nanoparticles and liposomes.

The composition can be formulated for any suitable routes of administration, including but not limited to, enteral, parenteral, systemic, and peripheral routes, such as intravenous, intramuscular, intraarterial, intrathecal, intracerebroventricular, intraventricular, intracapsular, intraorbital, intracardiac, intradermal, intranasal, intraperitoneal, intraparenchymal, transtracheal, subcutaneous, subcuticular, intraarticular, subpial, subarachnoid, intraspinal, intracerebral, oral, topical, and intrasternal route.

### Drug Development

This disclosure also contemplates utilization of R1β as a target for screening or designing candidate compounds for treating neurodegenerative diseases. Neuronal cells, neurons, animal models, or other appropriate in vitro, ex vivo, or in vivo systems can be contacted with test compounds to assay the compounds' ability to specifically decrease a level or activity of R1β to screen for potential drug candidates. For example, engineered cells containing R1β gene reporter constructs or expressing R1β tagged with a detectable or selectable marker (e.g., iPSC stably transformed with an expression vector or modified using gene editing techniques such as CRISPR) can be differentiated into neuronal cells and contacted with test compounds. Expression of the reporter constructs or level of the tagged proteins can be determined to identify compounds capable of specifically modulating R1β level or activity. Other PKA signaling events such as CREB phosphorylation can also be assayed to identify drug candidates.

### Unexpected Results and Advantages

In the central nervous system, CREB pathway activation is critical for the regulation of many essential neuronal processes including cell survival, development, neuroinflammation, and synaptic plasticity. The data described herein supports the role of dysfunctional PKA activation as a driver of decreased pCREB in FTD/ALS pathogenesis. First, it was found that the homeostatic balance between PKA R1β and Ca subunits was altered in C9 HRE neurons, reducing the ability of Ca to translocate to the nucleus to activate CREB. Second, overexpression of R1β in healthy control neurons was sufficient to decrease pCREB levels under basal conditions. Third, similar alterations to the PKA/pCREB signaling axis in postmortem C9 patient brain tissue were uncovered, where higher levels of R1β expression in MAP2-positive motor cortex neurons was associated with lower Ca and pCREB nuclear levels

Driving increased R1β levels, both transcriptional and posttranslational mechanisms could be proposed. In fact, it is known that upon persistent neuronal stimulation, PKA R subunits are degraded via the ubiquitin-proteasome system (UPS), sustaining Co enzymatic activity. Impaired UPS activity, which has been proposed as a disease-driving mechanism in FTD/ALS, would thus result in PKA R accumulation and reduced CREB activation, while increasing proteasome function could be proposed to restore normal cellular signaling. However, attempts to detect signs of UPS dysfunction, such as accumulation of aggregation-prone TDP-43 or other ubiquitinated proteins, in young C9 HRE neurons, have failed. Regardless, the data disclosed herein demonstrate that chronic treatment with rolipram, which has been used extensively for long-term treatment of in vitro and in vivo models, led to sustained higher cAMP levels and PKA activation, and significantly reduced R1β/Ca levels in HRE neurons. Importantly, it was shown that modulation of cAMP levels upstream of PKA activation via rolipram not only remedied the PKA subunit imbalance but also restored dendritic branching and postsynaptic proteins to control levels.

On the other hand, the use of PDE inhibitors has failed clinical trials despite promising results in preclinical models. Thus, simply increasing cAMP levels—particularly in advanced stages of the disease-would not be sufficient to maintain PKA activation as the excess R subunits would prevent its function. Directly targeting PKA R subunits to reduce their expression would allow for an enhanced bioavailability of PKA catalytic subunits to enter the nucleus and induce CREB activation, thereby promoting cellular health and neuronal function. Increasing the specificity of treatments could result in more potent responses while at the same time reducing potential side effects. The data described herein show that this could be accomplished by selectively targeting excessive PKA regulatory subunits directly, for instance by reducing cellular levels via RNA interference, antisense oligonucleotides (ASO), and/or with proteolysis targeting chimeras (PROTAC). Each of these strategies, by specifically reducing the levels of PKA regulatory subunit, could promote a more active PKA state thereby restoring the physiological activation of the CREB pathway.

This disclosure also shows that significantly lower levels of active (i.e. phosphorylated) CREB and PKA were found in both C9ORF72-ALS tissue and SOD1 mutant neurons in the motor cortex compared to control healthy samples. Mutations in the SOD1 gene are one of the most common causes of familial ALS (10-20% cases), second only to mutations in the C9ORF72 locus (30-40% cases). Together, these two mutations account for 40-60% of all familial ALS patients. While many aspects of pathology are not conserved between SOD1 and C9ORF72 mutant cases, such as the presence of TDP-43 aggregates and the prevalence of FTD-like symptoms, CREB pathway dysfunctions appear to be one of a few common signs of disease. Overall, the new data described herein support targeting CREB activation via modulating PKA regulatory subunit levels as an impactful approach to mitigate disease symptoms, with the potential to benefit a large number of ALS patients.

The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All patents, patent applications, and other publications (e.g., scientific articles, books, websites, and databases) mentioned herein are incorporated by reference in their entirety.

### Example 1

Dendritic arborization and synaptic complexity is diminished in C9ORF72 HRE iPSC-derived neurons.

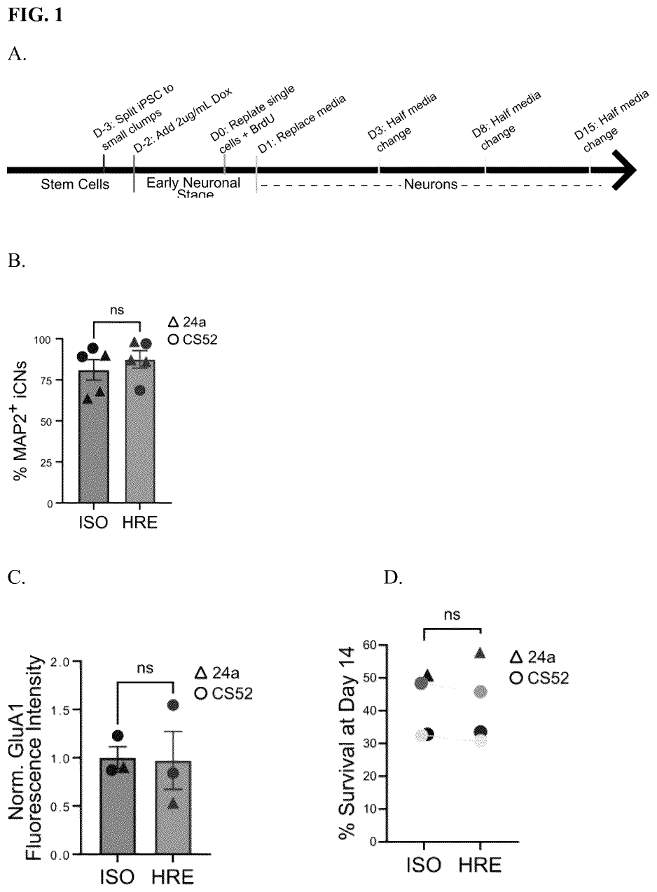

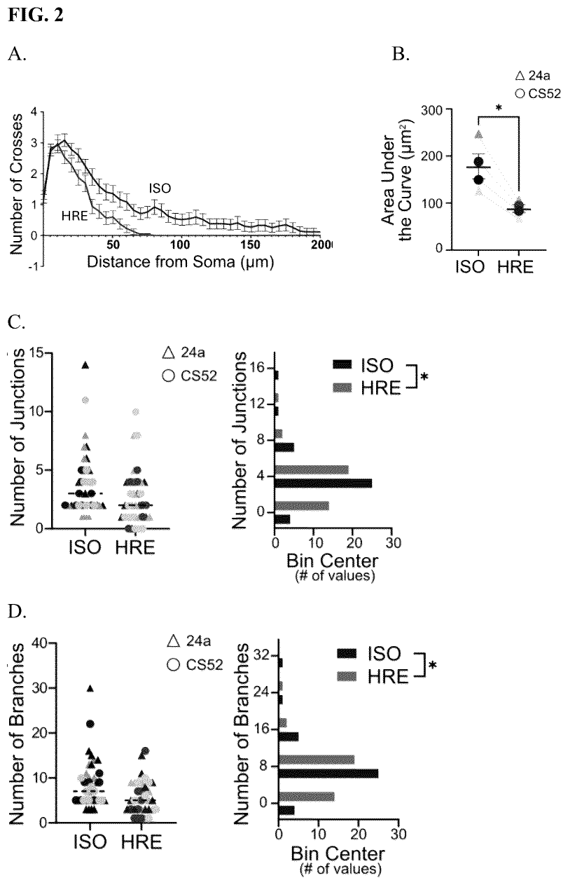

To determine whether C9 HRE causes early changes to neuronal dendritic and synaptic maturation that may be relevant to FTD/ALS pathogenesis, dendritic branching and synaptic markers expression in cortical-like neurons (CNs) differentiated from two independent patient-derived iPSC lines (i.e. 24a and CS52) carrying the GGGGCC repeat expansion (HRE) and their isogenic control lines (ISO), where the expansion has been reverted to normal ranges, were investigated (Table 1). To induce neuronal differentiation, the i³ approach was used, based on the transient ectopic expression of the transcription factor Neurogenin2 to induce rapid and uniform neuronal differentiation, as previously shown (FIG. 1). See Sirtori et al., bioRxiv [Internet]. 2024; 1 (doi.org/10.1101/2024.02.01.578318); Sirtori et al., Acta Neuropathol Commun. 2024; 12 (1): 69. HRE and ISO neurons from both iPSC lines differentiated with similar efficiency to mature MAP2-positive neurons with membrane expression of the glutamate AMPA receptor (FIGS. 1, B and C). Survival analyses of ISO and HRE neurons also showed similar viabilities at 14 days in vitro across all lines (FIG. 1, D).

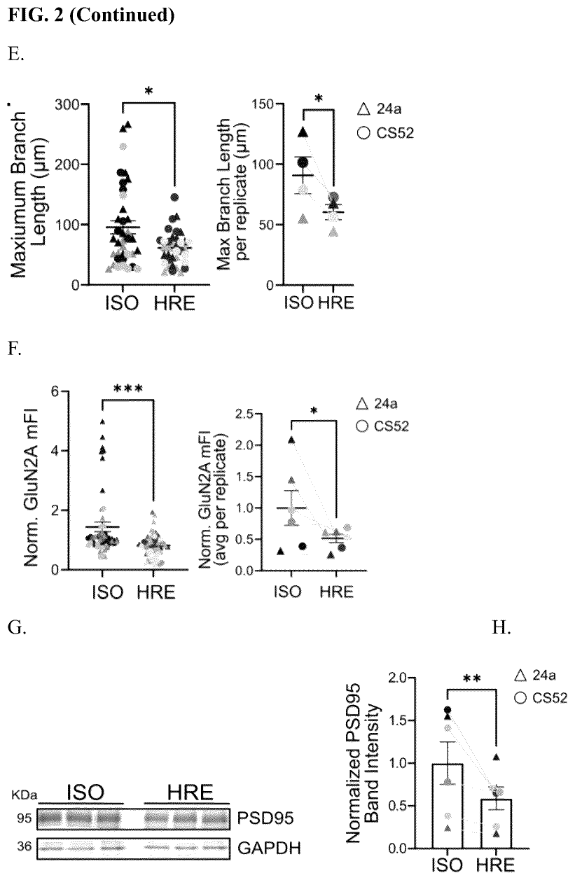

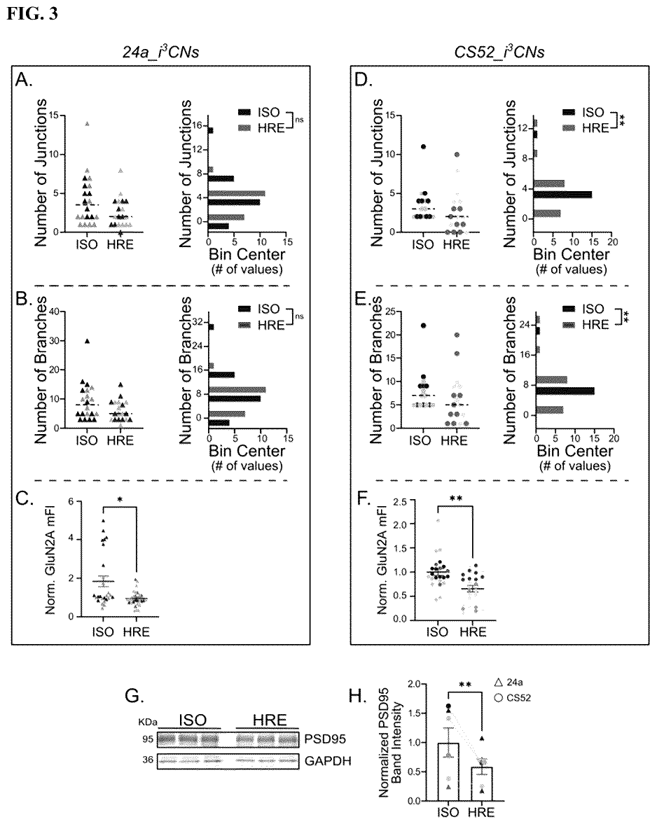

To characterize changes to the complexity and branching of the dendritic arbor, 2-week-old HRE and control neurons were stained for the cytoskeletal protein MAP2, and immunofluorescence images were subjected to Sholl and skeleton analyses (FIG. 1 and FIG. 2). Under these conditions, it was found that a significant reduction in the number of dendrite crosses by Sholl analysis in HRE compared to ISO i³CNs, which resulted in a significant reduction of the calculated area under the curve (FIGS. 2, A and B). HRE neurons were also characterized by a significant reduction in the number of dendritic junctions and branches and in the maximum length of these branches (FIG. 2, C-E). To determine if these alterations were also associated with changes to postsynaptic structures, this study investigated the levels of membrane expression of the glutamate NMDA receptor subunit GluN2A, as well as the overall levels of the abundant postsynaptic density 95 (PSD95) protein, which is responsible for the stabilization, recruitment, and trafficking of NMDA receptors to excitatory synapses. Consistent with the branching defects, it was found that C9 HRE neurons had a mild but significant reduction in dendritic GluN2A surface expression (FIG. 2, F) as well as lower levels of PSD95 (FIG. 3, G-H) compared to ISO counterparts. Altogether, the data indicate that the presence of the C9 HRE leads to a reduction in dendritic branching and synaptic proteins, similar to what has been observed in postmortem tissues from FTD/ALS patients.

### Example 2

CREB activation is reduced in C9 HRE i³CNs.

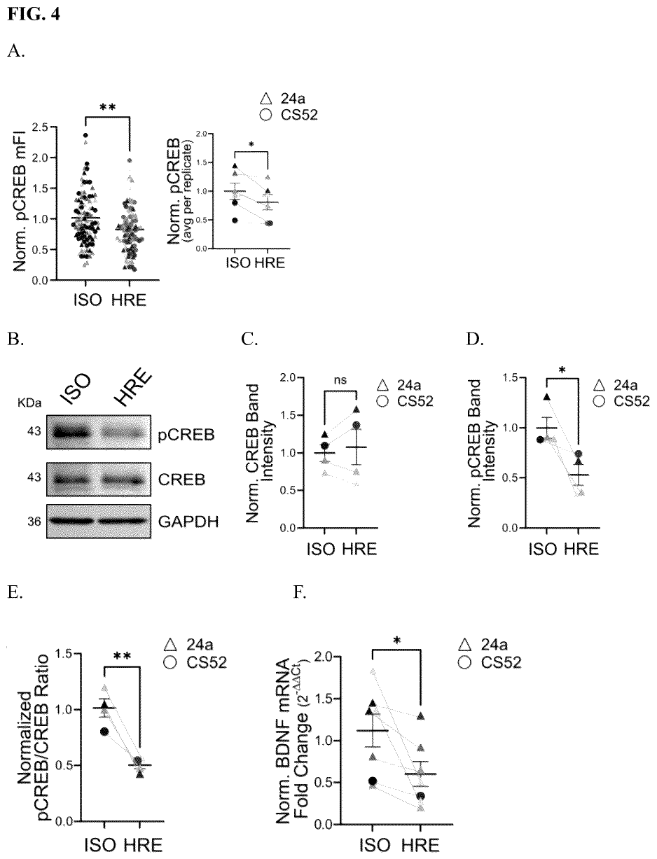

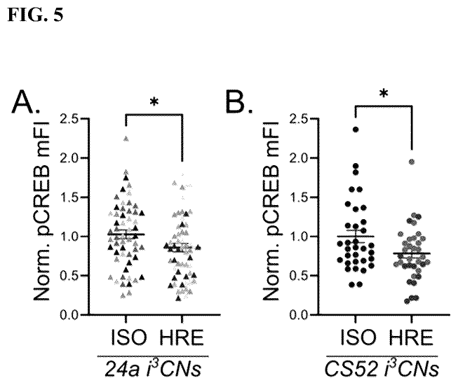

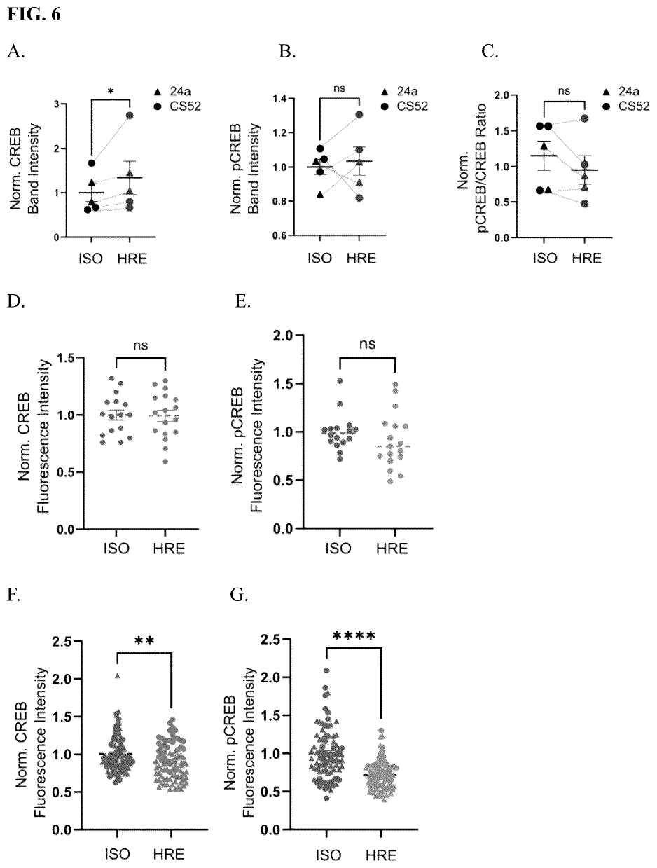

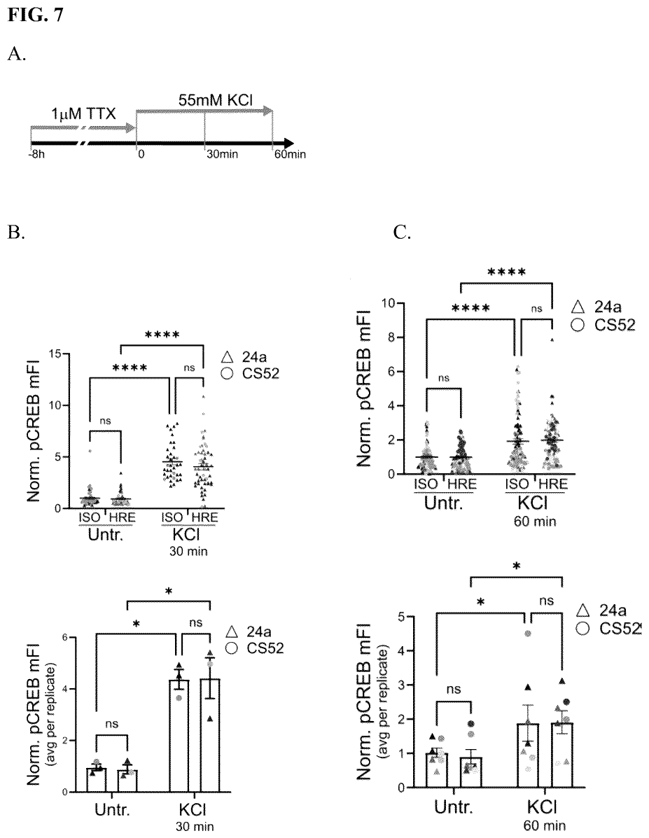

Since the CREB pathway is one of the major modulators of dendritic branching and synaptic maturation, it was investigated whether alterations to CREB activation could underlie the changes in dendritic arborization observed. Thus, this example quantified the levels of phosphorylated (i.e., active) CREB (pCREB) under basal conditions in HRE and ISO i³CNs (FIG. 4 and FIG. 5). Immunofluorescence analysis demonstrated a significant reduction in nuclear levels of pCREB protein in mutant neurons compared to isogenic controls (FIG. 4, A), which was confirmed by western blot analyses (FIG. 4, B-D). While overall CREB levels were similar between ISO and HRE neurons, it was found that the pCREB to CREB ratio was significantly lower in mutant cells (FIG. 4, E), indicating that a change in CREB posttranslational activation rather than lower protein expression levels was causing the observed defect. Functionally, pCREB reduction was associated with lower levels of the BDNF mRNA (FIG. 4, F), a well-known CREB target that plays key roles in regulating neuronal growth and synaptic plasticity. Decreased pCREB levels were also found in C9 HRE cells at early neuronal stages (i.e. 3 days post-induction), though not at the stem cell state (FIG. 6), indicating that pCREB reduction occurs only after cells commit to the neuronal lineage fate. Overall, these data suggest that the activation of the CREB pathway is impaired in HRE neurons.

### Example 3

CREB PKA-dependent activation is impaired in C9 HRE neurons.

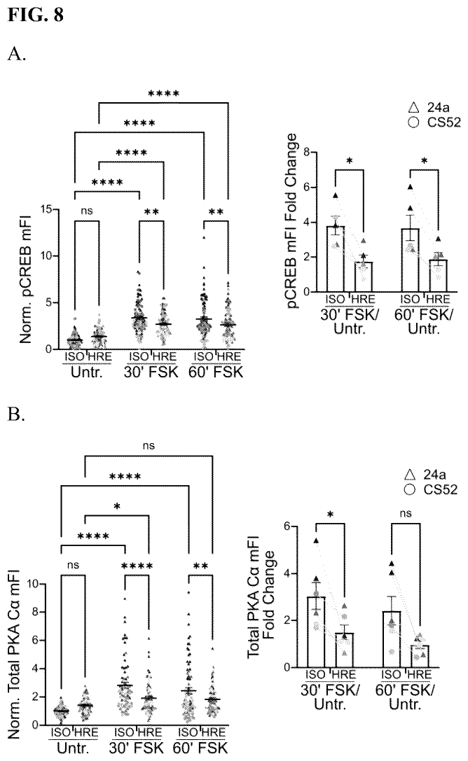

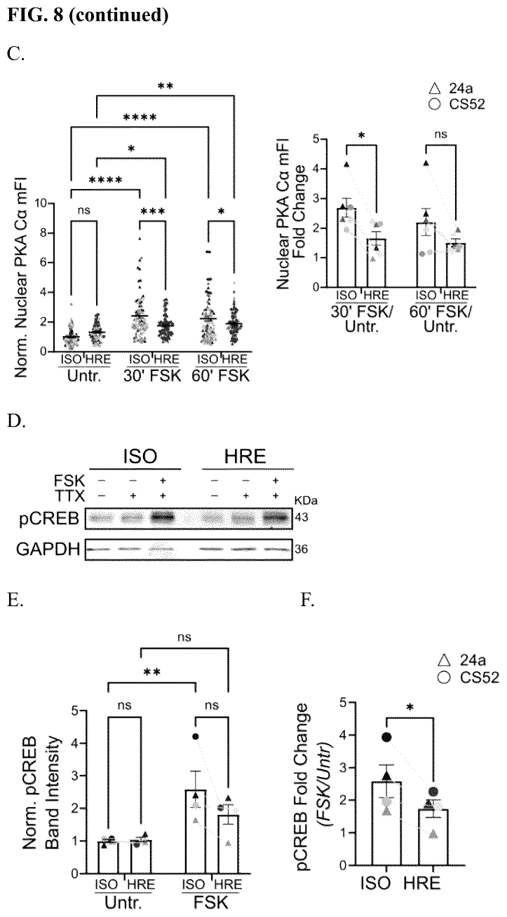

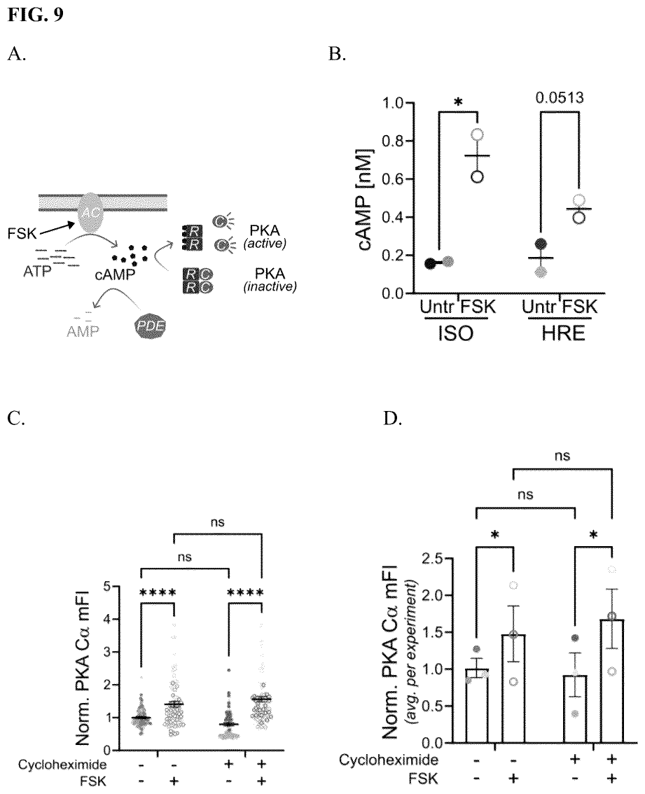

To tease out the molecular determinants underpinning reduced pCREB levels in HRE i³CNs, this example stimulated neurons with compounds to forcibly induce CREB phosphorylation via two separate pathways. This study chose potassium chloride (KCl) and forskolin (FSK) as these have been previously shown to induce CREB activation via the calcium-dependent induction of the calcium-calmodulin kinase IV (CamKIV) or via the cAMP-dependent activation of protein kinase A (PKA), respectively. Importantly, these compounds do not rely on receptor-dependent signaling, thereby eliminating potential confounding variables due to differential expression of such receptors between mutant and control cells. To further reduce potential variability in experimental output due to spontaneous activity, this study pre-treated neurons with 1 μM tetrodotoxin (TTX), a selective inhibitor of voltage-gated sodium channels. Under these conditions, it was found that KCl treatment for either 30 minutes or 1 hour led to a significant increase in pCREB levels in treated neurons compared to untreated controls (FIG. 7). However, no difference in the response of HRE versus ISO neurons was evident, as the levels of pCREB at each time point were comparably elevated (FIG. 7, B-C), suggesting that this specific pathway is not altered in mutant neurons. In contrast, stimulation with FSK for either 30 or 60 minutes, which led to a corresponding increase in cellular cAMP levels (FIG. 9, A-B), raised pCREB levels in HRE i³CNs significantly less than in ISO controls (FIG. 8, A). This differential response to FSK treatment was also validated by western blotting, which confirmed the significantly diminished activation of CREB in HRE neurons (FIG. 8, D-F).

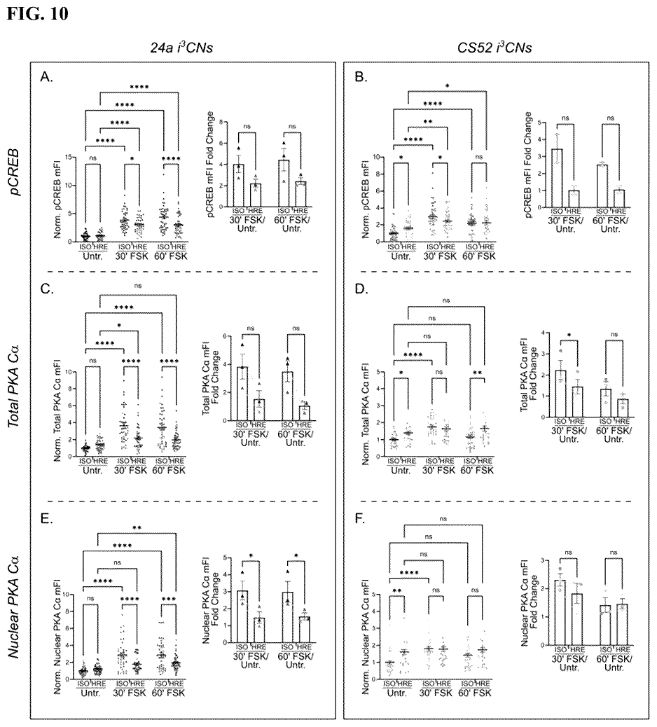

Since FSK-mediated CREB phosphorylation depends on the activity of PKA, this study investigated whether the altered response to FSK in HRE neurons was caused by reduced activation of this kinase. PKA exists as a hetero tetramer composed of a regulatory (R) dimer and two catalytic (C) subunits. cAMP binding to the holoenzyme leads to the separation of the regulatory dimer from the catalytic subunits, which thus become active. While the PKA Ca antibody used in this example was not selective for the active enzyme, it was found that it could be reliably used as a proxy for “active” Cα (see FIG. 9). In fact, even after short FSK stimulations, the result showed a rapid increase in fluorescence intensity that was insensitive to protein synthesis inhibitors and depended on increased epitope availability. In addition, given that only the active Ca monomers can enter the nuclear compartment to activate downstream targets, this study selectively quantified nuclear Ca levels, thus overcoming the limitations of relying on increased antibody accessibility to the protein antigen. Using this approach, it was found that both total and nuclear levels of the Ca subunit of PKA were significantly reduced in mutant neurons following FSK stimulation (FIG. 8, B-C). These defects were observed in neurons differentiated from both iPSC lines with comparable magnitude (FIG. 10). Of note, it was found that while TTX treatment in 24a i³CNs abolished any difference in the basal pCREB and PKA Cα levels between HRE and ISO neurons, this was not the case in CS52 neurons, where TTX treatment resulted in slightly elevated pCREB and Ca levels in mutant cells (FIG. 10, B, D, F). Regardless, C9-mutant neurons from both lines did not activate the PKA-pCREB pathway in response to FSK stimulation. Overall, the differential response of C9 HRE neurons to KCl and FSK stimulation indicated that the observed CREB dysfunction is specific to the cAMP-PKA-CREB signaling branch, and that it could be directly caused by the failure of induction of PKA.

### Example 4

PKA inhibition leads to altered cellular morphology in control neurons.

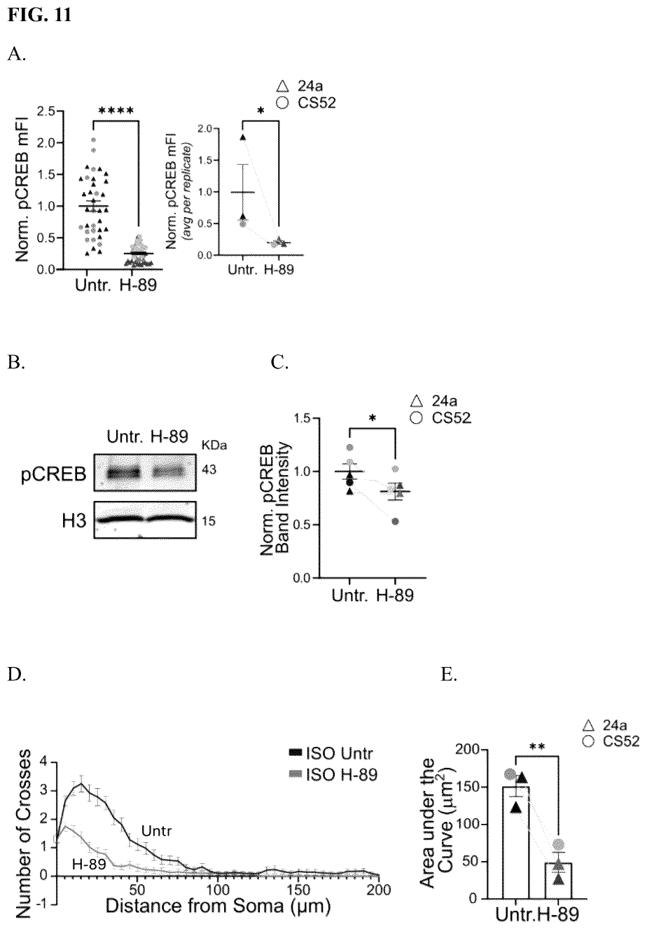

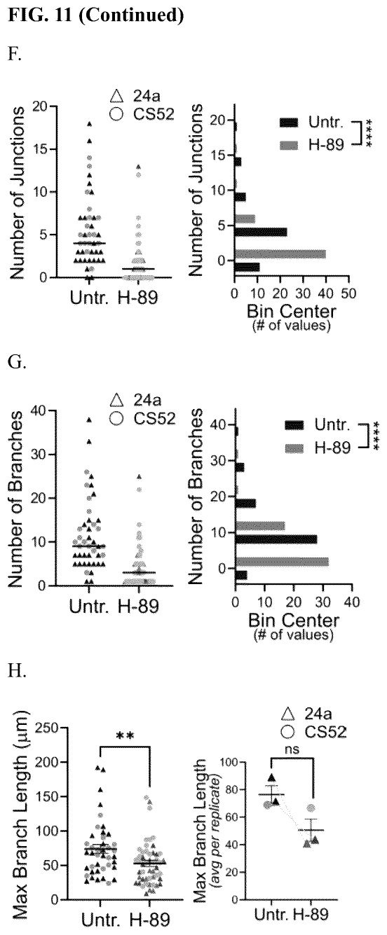

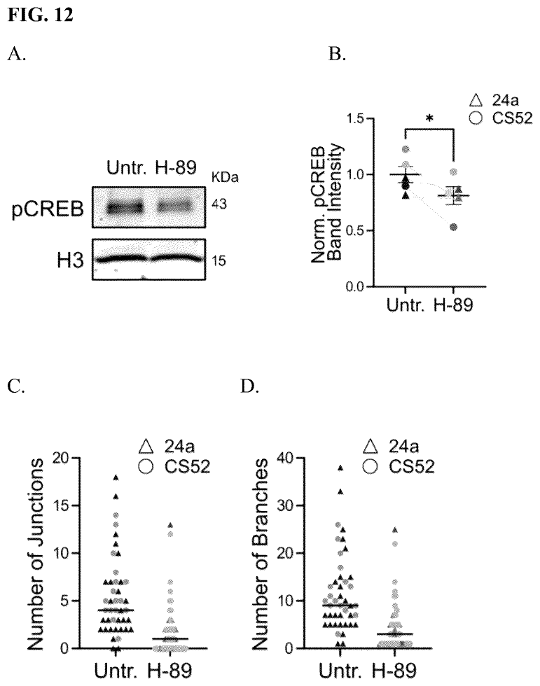

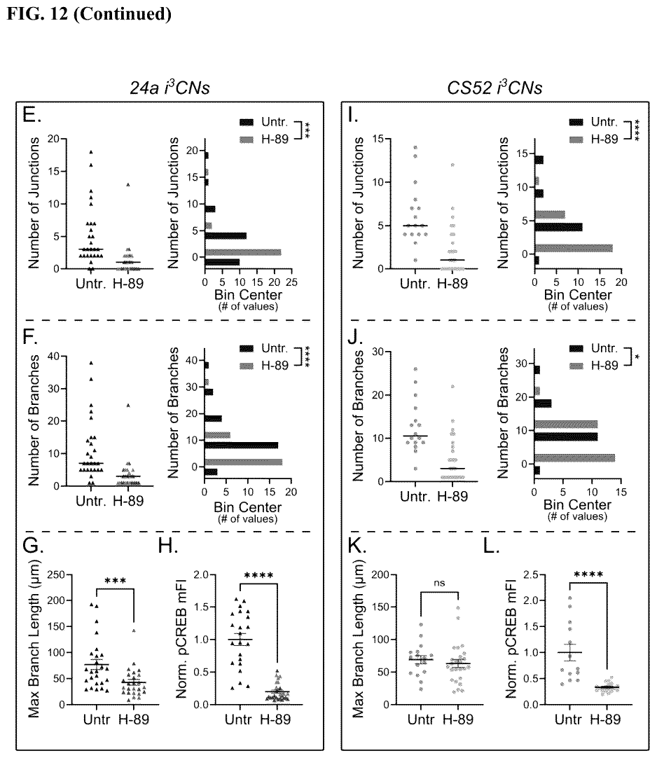

While it is known that one of the main targets of CAMP is PKA, this essential second messenger can activate a host of downstream cellular effectors that regulate diverse aspects of cellular function. To test whether the effects observed following FSK treatment described herein were linked to PKA activity specifically rather than another molecular player, ISO neurons were treated with 10 μM H-89, a PKA-specific inhibitor (FIG. 11 and FIG. 12). After 14 days of treatment, it was found that H-89 treatment caused a reduction in the levels of pCREB in ISO neurons, as demonstrated by both immunofluorescence and western blot assays (FIG. 11, A and FIG. 12, A-B). This decrease was also associated with reduced dendritic arborization and complexity (FIG. 11, B-E and FIG. 12, C-L), phenocopying the defects observed in HRE neurons (see FIG. 2). Overall, these data suggest that altered activation of the PKA-CREB branch of the cyclic nucleotide signaling pathway is a main driver of cellular pathology in HRE neurons.

### Example 5

Dis-homeostasis of PKA regulatory and catalytic subunits reduces PKA activation.

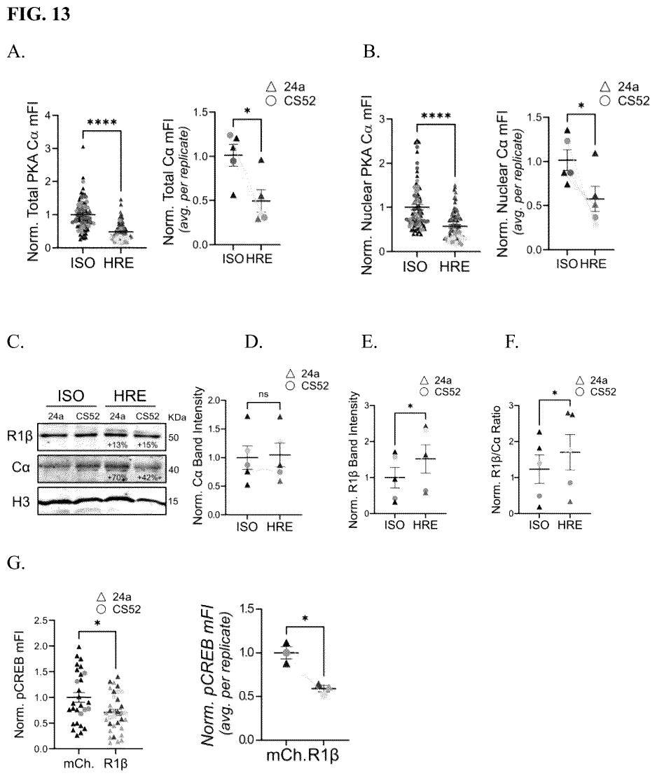

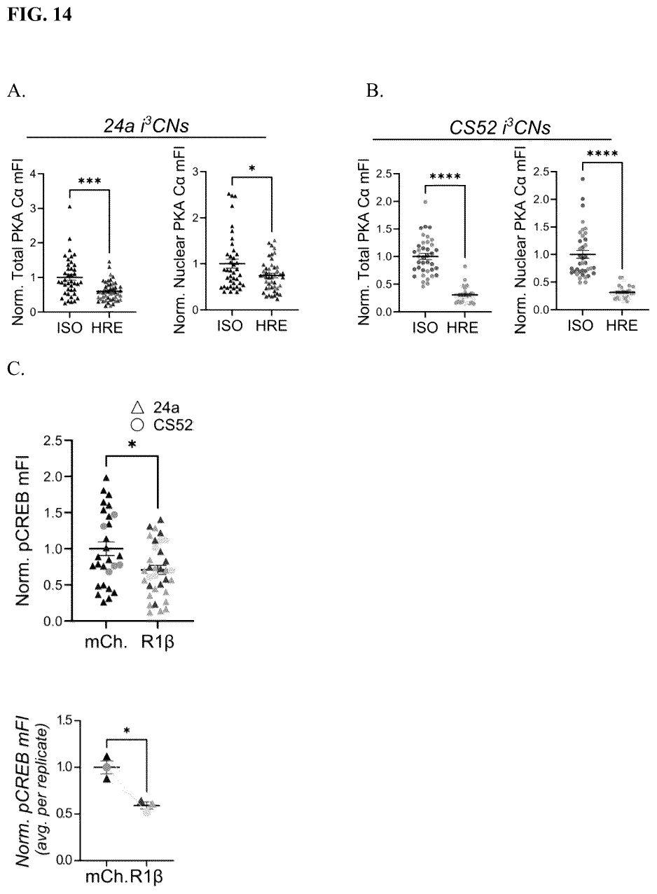

Since the data disclosed herein indicated that changes in PKA activation are causative for the diminished pCREB levels in C9 neurons following FSK stimulation, it was decided to further investigate PKA activation and levels of both regulatory (R) and catalytic (C) subunits in HRE i³CNs under basal conditions (FIG. 13). First, it was confirmed that the levels of active PKA in C9 HRE neurons were lower compared to ISO controls even in the absence of direct FSK stimulation (FIG. 13, A and FIG. 14). To assess whether Ca reduction reflected an overall decrease in the levels of the PKA holoenzyme, this study performed western blot assays on whole cell lysates from HRE and ISO neurons. While the use of the Ca-specific antibody allows to quantify the levels of the active enzyme in IF assays (FIG. 9, D), western blot approaches, due to the use of denaturing conditions, can only measure overall levels of both regulatory and catalytic subunits, independent of their activation state. These assays focused on the PKA regulatory subunit 1β (R1β), which is most abundantly expressed in neurons. Interestingly, while overall protein levels of the Ca subunit did not differ between ISO and HRE neurons (FIG. 13, C-D), this study showed a significant upregulation of the regulatory R1β subunit, which resulted in a higher R to C ratio in C9 HRE neurons (FIG. 13, E-F). The reduction in active Ca observed via IF and the increased expression of R1β identified via western blot analyses may indicate a homeostatic dysregulation of PKA complex subunits, wherein an excess of regulatory subunits may lead to a down-regulation in the cellular availability of active (i.e. unbound) catalytic subunits, triggering a reduction in the downstream phosphorylation of PKA targets, including CREB. To test this possibility, overexpression of untagged PKA R1β subunit or mCherry as a control in ISO neurons via lentiviral transduction was used (FIG. 14, C). While R1β overexpression resulted in increased cell toxicity, increasing its cellular levels was sufficient to significantly reduce CREB activation in otherwise healthy control neurons, suggesting that the upstream alteration of PKA activity is a key driver of pCREB-dependent cellular defects.

### Example 6

PKA and CREB alterations are a feature of C9-ALS patient's postmortem tissues.

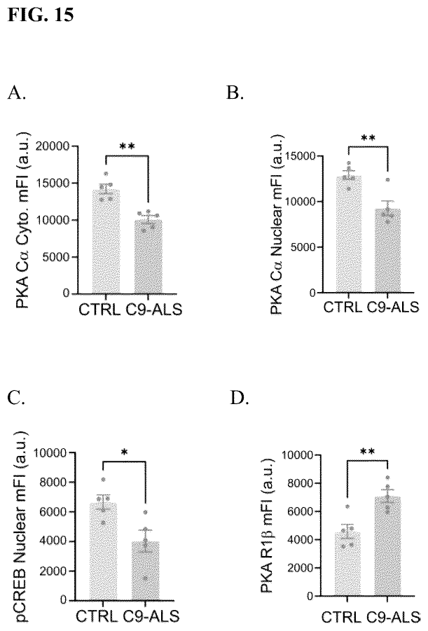

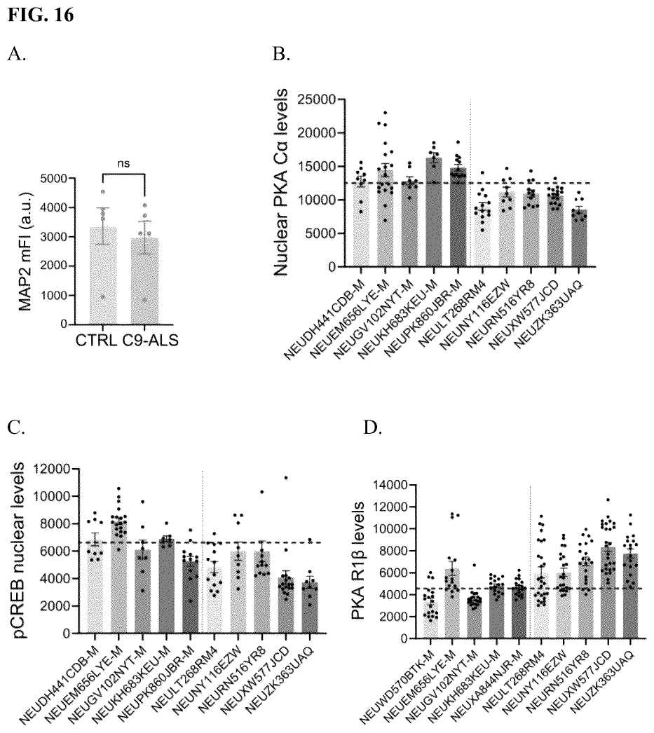

The observation that CAMP-PKA-CREB signaling is altered in neurons differentiated from two independent C9-mutant iPSC lines posed the question of whether these defects are also relevant in the context of human pathology, and therefore observed in patients' postmortem tissue. To answer this important question, this study assessed the levels of pCREB, PKA Ca and R1β in motor cortex of five ALS patients carrying C9ORF72 mutations compared to 5 non-neurological controls (Table 2). While a clear nuclear staining for pCREB as well as strong signal in both the cytoplasm and nucleus for PKA Ca was evident in each control sample, C9-ALS neurons had significantly lower levels for both markers (FIG. 15, A-C and FIG. 16). As expected, given their functional and molecular connection, pCREB and nuclear Ca levels showed a strong in-cell correlation. Interestingly, control and C9-ALS neurons clearly clustered separately, with C9-ALS neurons showing lower levels for both markers (data not shown). By contrast, cellular levels of MAP2, which labels the microtubule network and is not directly dependent on CREB activity, were unchanged between C9-ALS and controls, showing no obvious correlation with Ca nuclear levels (FIG. 16, A-B). It was investigated whether pCREB and Ca reduced expression were also associated to higher R1β levels. Similar to what was observed in cultured neurons, it was found that C9-ALS tissues had increased expression of R1β compared to controls (FIG. 15, D). Overall, these data suggest that changes to the CAMP-PKA-CREB pathway may be relevant drivers of disease in ALS patients.

### Example 7

Modulating CAMP levels rescues PKA imbalance and pCREB levels.

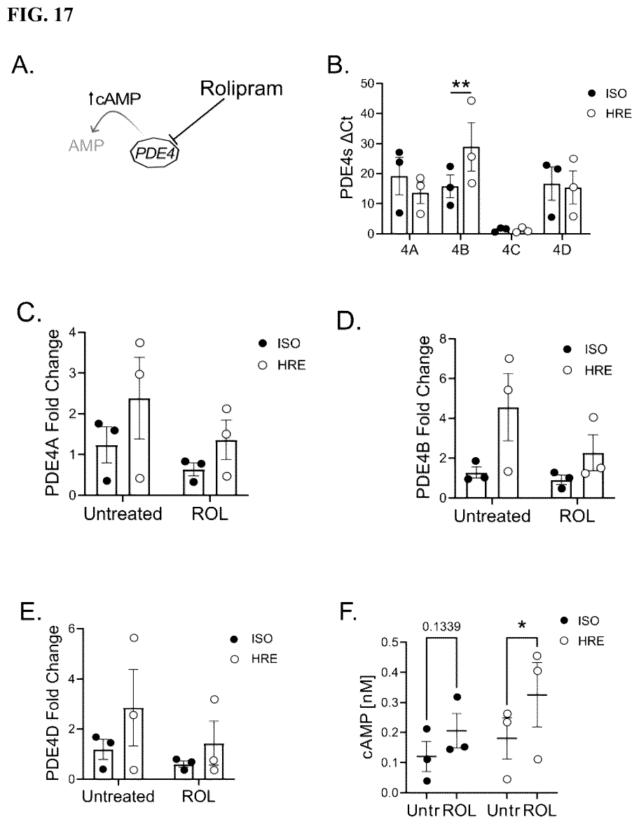

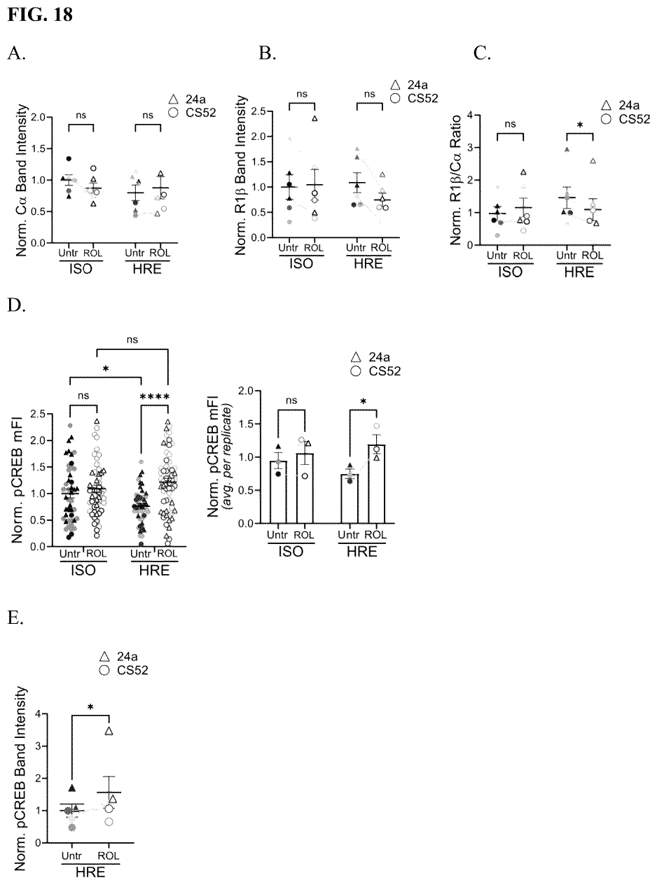

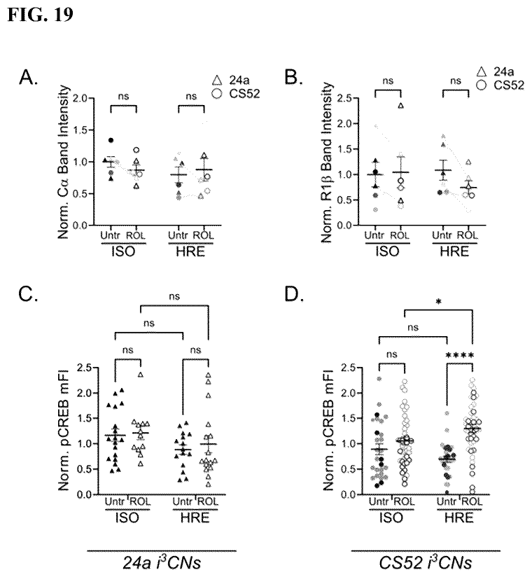

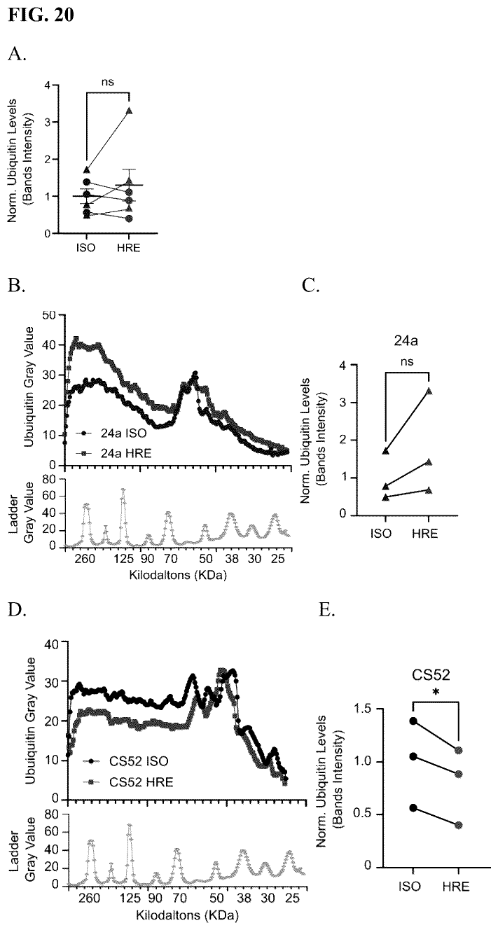

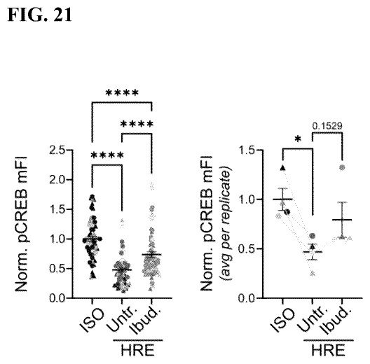

It is well established that prolonged cellular stimulations and high levels of CAMP lead to the long-term induction of PKA activity by promoting the proteasome-dependent degradation of PKA regulatory subunits. It was thus hypothesized that chronic elevation of cAMP levels in C9 mutant neurons could result in a rebalancing of the levels of PKA's C and R subunits, leading to a functional rescue of CREB activity. To test this possibility, this example treated i³CNs chronically for 2 weeks with rolipram (ROL), a selective inhibitor of phosphodiesterase-4 (PDE4), the main enzyme responsible for degrading cAMP to AMP (FIG. 17). Q-PCR analyses confirmed all four PDE4 isoforms were expressed at similar levels in HRE and ISO neurons, apart from 4B which was slightly elevated in HRE cells compared to ISO controls (FIG. 17, A-B). Chronic treatment with ROL caused a significant increase in intracellular cAMP levels, accompanied by a small but not significant decrease in the expression of all PDE4 isoforms (FIG. 17, C-F). The effects of ROL treatment on the levels of PKA subunits was evaluated (FIG. 18 and FIG. 19). No significant changes in total Ca or R1β levels in ISO neurons were found, suggesting rolipram has a limited effect in healthy cells. In contrast, chronic treatment with ROL in HRE neurons led to a slight increase of Ca levels accompanied by a slight decrease in the levels of R1β (FIG. 19, A-B). While these changes did not reach statistical significance per se, when the ratio of R1β/Ca levels were calculated, it was significantly reduced in HRE neurons (FIG. 18, A). These data suggested ROL could indeed restore PKA homeostasis, possibly by promoting R1β degradation via the ubiquitin proteasome system, which did not appear dysfunctional under our experimental conditions (FIG. 20). Changes in PKA subunit levels resulted in the rescue of CREB activation in HRE neurons, as shown by both IF (FIG. 18, 7 B-C and FIG. 20, B-C) and WB assays (FIG. 18, D), while no significant change was detectible in ISO cells. Of note, chronic treatment of neurons with ibudilast, an unrelated and non-selective PDE inhibitor currently in clinical trial for use in ALS and other neurodegenerative diseases, led to a more limited rescue on pCREB levels (FIG. 21), suggesting that targeting PDE4 isoforms could be a more specific approach to restore PKA-CREB activity in C9-FTD/ALS. See Angelopoulou et al., Molecules, 2022; 27 (23).

### Example 8

cAMP modulation restores dendritic complexity in C9 HRE neurons.

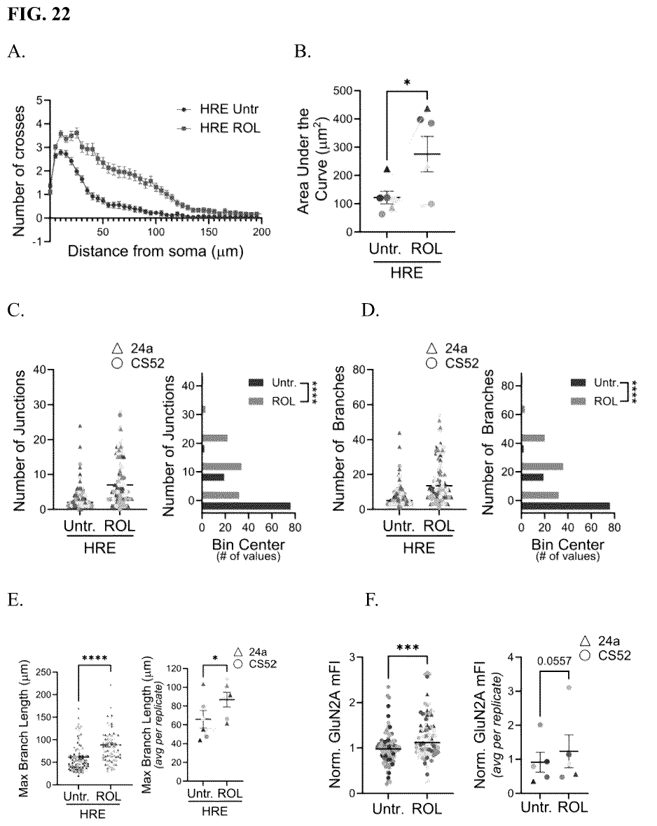

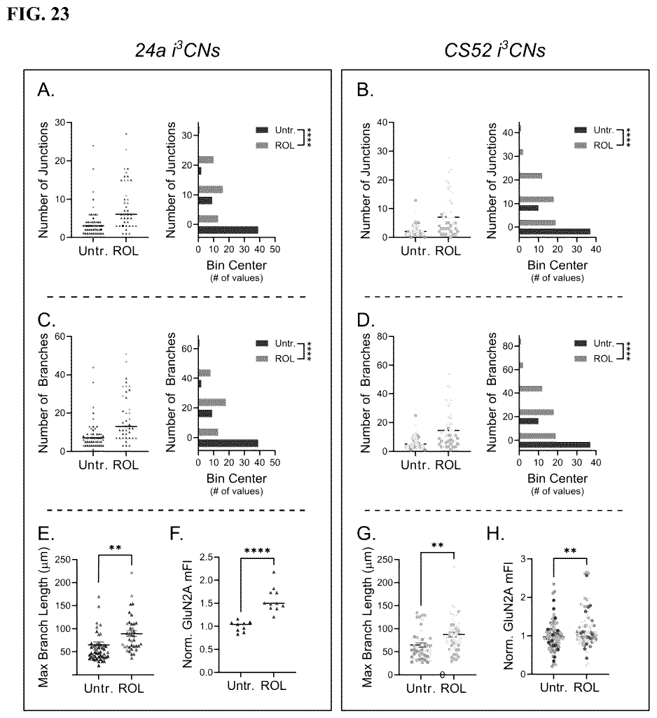

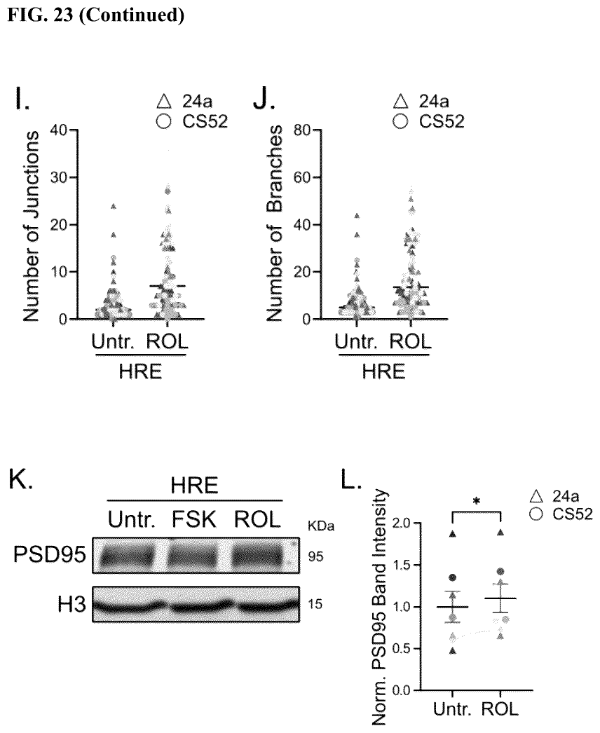

Since chronic treatment with ROL was able to restore the homeostatic ratio of PKA subunits and CREB activation, whether this approach could also rescue decreased dendritic branching and post-synaptic protein levels observed in HRE neurons was investigated. Neurons with ROL were chronically treated for two weeks, and dendritic morphology based on MAP2 staining in HRE neurons compared to untreated controls were evaluated (FIG. 22 and FIG. 23). Interestingly, ROL treatment significantly improved dendritic arborization, the number of dendritic branches, and the maximum dendrite branch length, as indicated by Sholl and skeleton analyses (FIG. 22, A-E). Treatment with ROL also led to a small but significant increase in PSD95 levels (FIG. 23, K-L) and of the NMDA receptor subunit GluN2A (FIG. 22, F). Altogether, these data suggest that modulating cAMP levels can not only influence PKA subunit homeostatic balance but, importantly, can lead to the rescue of CREB activation and downstream functional output.

### Example 9

PKA and CREB alterations are a feature of SOD1 mutant neurons.

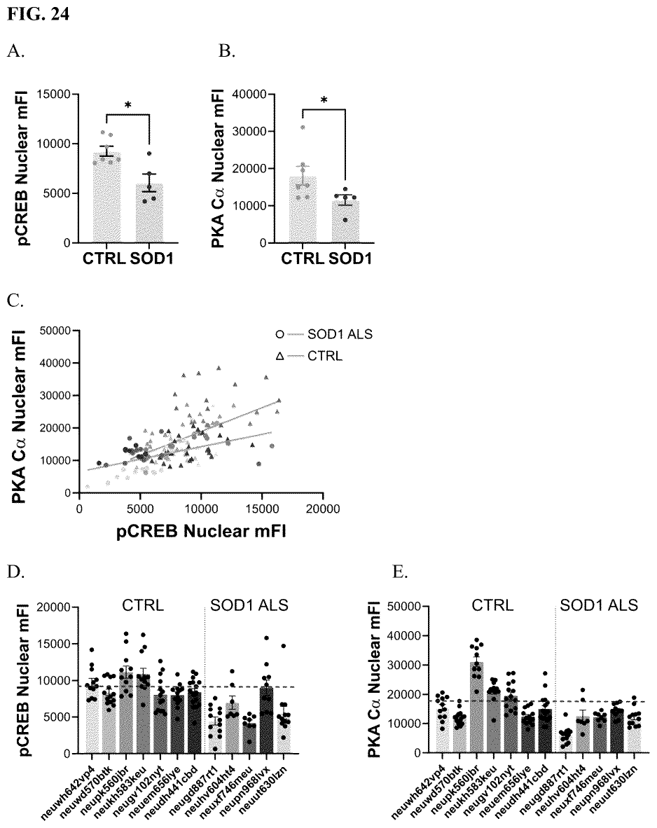

Similar to what was observed in C9ORF72-ALS tissue, it was found that SOD1 mutant neurons in the motor cortex had significantly lower levels of active (i.e. phosphorylated) CREB and PKA compared to control healthy samples (FIG. 24, A-B). While the levels of pCREB and PKA correlated strongly in control neurons as expected, given the mechanistic link between PKA activity and CREB phosphorylation, that correlation was lost in SOD1 ALS samples (FIG. 24, D). Finally, patient-level data (FIG. 24, D-E) confirms the phenotype is present in all samples examined.

### Example 10

### Materials and Methods

Cell Culture, iPSC Differentiation, and Treatments

Human induced pluripotent stem cell (hiPSC) lines detailed in Table 1 were cultured and differentiated as previously described. See Sirtori et al., Acta Neuropathol Commun. 2024; 12 (1): 69. Detailed methods are described in the Supplementary Methods. To induce CREB activation, cells were treated with 55 mM potassium chloride (KCl) and 5 μM forskolin (FSK) for 30 or 60 minutes, while chronic treatment with H-89 (10 μM), rolipram (2 μM) or ibudilast (10 μM) was used to inhibit PKA or increase CAMP cellular level, respectively.

### Cells and Tissue Processing and Imaging

Cells grown on glass coverslips were fixed with 4% paraformaldehyde processed for immunofluorescence as described. See Sirtori et al., Acta Neuropathol Commun. 2024; 12 (1): 69. Human postmortem motor cortex paraffin sections were obtained from TargetALS biorepository and processed as described⁴⁶. Samples were imaged using a widefield microscope (Leica DMi8 Thunder) as Z-stacks (0.21 μm step size) using a ×63 lens and deconvolved with an adaptative blind deconvolution algorithm (Autoquant X3, Media Cybernetics). All image analyses were performed using Fiji/ImageJ software. Additional information can be found in the Supplementary Methods and Table 3.

### RNA and Protein Extraction

Total RNA was extracted from neurons using the RNeasy mini kit (Qiagen). Total protein lysate was obtained using lysis buffer (150 mM NaCl, 20 mM Tris pH 8, 1% Triton X-100) supplemented with protease inhibitor (Sigma Aldrich, 11836153001) and phosphatase inhibitors (Sigma Aldrich, 4906837001). Detailed information can be found in Table 3 and in the Supplementary Methods.

### Statistical Analyses

Parametric or non-parametric tests were performed depending on normality of the datasets. For datasets with un-matched points (i.e. individual cells) unpaired two-tailed t tests, Mann Whitney tests, ordinary one-way or ordinary two-way ANOVAs were used. Data where points were matched between the groups (i.e. averaged biological replicates, western blot and qPCR data) were analyzed by ratio paired t tests, repeated measures (RM) one-way or two-way ANOVAs. Discrete categorical data were binned into 5 groups using appropriate bin centers and the distributions were analyzed with Fisher's exact test. In all graphs, data obtained from independent replicates are indicated using different shades, while data obtained from the two iPSC lines used are indicated by triangles (24a ISO and HRE) or circles (CS52 ISO and HRE).

### Supplementary Methods

Cell Culture and iPSC Differentiation

Human induced pluripotent stem cell (hiPSC) lines detailed in Table 1 were cultured in Stemflex media (Gibco, A3349401) on Matrigel (Corning™ 356234) coated plates and were passaged every 4-6 days with 0.5 mM EDTA in Ca²⁺ and Mg²⁺-free 1×PBS (Thermo Fisher Scientific). Induced, integrated, and isogenic cortical neurons (13CNs) were generated by inducing stem cells to differentiate with an induction media (DMEM/F12 with HEPES; Gibco, 11330032) containing doxycycline (Sigma Aldrich, D52071G) to activate the TO-hNGN2-BSD-mApple (NGN2) cassette (AddGene Plasmid #124229) that had been integrated in the CLYBL safe harbor locus via Crispr/Cas9 gene editing, and N2 (Gibco, 17502048), NEAA (Gibco, 11140050), Glutamax (Gibco, 35050079), rock inhibitor (Tocris, 1254), and compound E (Millipore Sigma, 565790500UG). Mature neurons were obtained by plating single cells on 1:1 poly-D-lysine/poly-L-ornithine (PDL/PLO; Sigma Aldrich, P7405/P3655) and culturing in Neurobasal media (Gibco, 21103049) supplemented with N2, B27 (Gibco, 17504044), NEAA, Glutamax, and Laminin (Corning, 354232). Neurons were aged with half media changes and sterile water to compensate for evaporation in the incubator each week since their establishment in neuron media until two-week sampling timepoint. Survival analyses were performed on neurons plated in 96-well plate format and imaged over time via a long-term cell imager (IncuCyte Zoom3). The number of mApple-positive cells was quantified on 4 fields of view per well and averaged from 3 wells per differentiation.

HEK-293 cells were cultured in high glucose DMEM media (Gibco, 11965092) with 10% FBS (R&D Biosystems, S11150) and plated on coverslips coated with 1:1 PDL/PLO at a density of about 80,000 cells per well.

### Cell Treatments

Potassium chloride (KCl) and forskolin (FSK) were used to stimulate CREB activation via distinct pathways. Prior to stimulation with these compounds, cells were treated with 1 μM sodium channel blocker tetrodotoxin (TTX; Biotium) for 8 hours to silence action potentials. After 8 hours incubation with TTX, the cells were washed with 1×PBS before stimulation with compounds. To selectively stimulate the CamKIV branch of CREB activation, 55 mM KCl (Alfa Aesar) was added to neuronal media and was applied to cells for 30 minutes, 1 hour, and 6 hours. To selectively activate the PKA branch of CREB activation, 5 μM FSK (Cayman Chemicals) was introduced into neuronal media for 10, 30, and 60 minutes. To inhibit PKA enzymatic activity, cells were treated chronically with 10 μM H-89 (Selleck Chem) from day 0 until 14 days post differentiation. Modulation of PKA R levels was accomplished via chronic two-week treatment starting at day 0 of differentiation with 2 μM Rolipram (ROL; Tocris) or 10 μM ibudilast (Selleck Chem) added into neuronal media. As half of the neuron media was changed once a week until the two-week sampling timepoint, half of the compounds were added back into the media to maintain a consistent concentration. HEK-293 cells were treated with 10 μg/ml of protein synthesis inhibitor cycloheximide (Acros Organics) for 1 hour prior to stimulation with 5 μM FSK for 10 minutes. Cells were immediately fixed and processed for immunofluorescence analyses as detailed below.

### Viral Transduction

Lentiviral constructs to overexpression R1b PKA subunit (4.17×10⁸ TU/ml) or mCherry as control 2.69×10⁸ TU/ml) were purchased from Vectorbuilder. Human PRKAR1B (NM_002735.3) or mCherry coding sequences were cloned under the control of the EF1A promoter and followed by the WPRE element to optimize expression. EGFP, expressed under the control of the CMV promoter, was used as marker of efficient transduction. Viruses (1 μl/well) were added to differentiated neurons at day 1 post differentiation. Cells were then fixed at 14 days in vitro and processed for immunofluorescence as described above.

### Immunofluorescence

Cells grown on glass coverslips were fixed with 4% paraformaldehyde, permeabilized with 0.2% tween in 1×PBS, and blocked with 5% bovine serum albumin before hybridization with primary antibodies of interest: CREB (Cell Signaling), pCREB (Cell Signaling), catalytic subunit PKA (PKA[c]) (BD Biosciences), MAP2 (Thermo Fisher Scientific), and GluN2a (NeuroMab). Cells stained with GluN2a were not permeabilized before exposure to the antibody to allow for investigation into the cell surface-level expression. Secondary stains were performed with either Alexa 488, Rhodamine X, or Alexa Fluor 647 from Jackson Immunoresearch. Coverslips were mounted with Invitrogen ProLong Gold Antifade mountant with DAPI (Thermo Fisher Scientific) and were imaged at either 20× or 63× with 0.21 μm step sizes on a Leica DMi8 widefield fluorescent microscope equipped with a CMOS camera (DFC9000 GTC). After immunofluorescence image acquisition, 63× images were deconvolved with an adaptative blind deconvolution algorithm (Autoquant X3, Media Cybernetics). Detailed information on the antibodies used can be found in Table 3.

### Human Postmortem Processing and Immunofluorescence

Human postmortem motor cortex paraffin sections were obtained from TargetALS biorepository. Clinical data from the cases are summarized in Table 2. Sections were de-paraffinized in xylene (Sigma-Aldrich) and re-hydrated with scaling dilutions of ethanol (Thermo Fisher Scientific) to MilliQ water. Antigen retrieval was performed in 10 mM citric acid pH 6 with 0.01% Triton X-100 at 100° C. for 20 min. After an hour of cool-down, slides were washed with 1×PBS and incubated with blocking buffer (1% BSA, 5% FBS, 0.01% Triton X-100 in 1×PBS). Slides were incubated overnight at 4° C. with primary antibodies (see Table 3) and at room temperature for 2 h with secondary antibodies. Slides were mounted onto a glass slide using Prolong Gold mounting medium (Thermo Fisher Scientific) and imaged using a widefield microscope (Leica DMi8 Thunder) equipped with a cooled CMOS camera (DFC9000 GTC). Images were acquired as Z-stacks (0.21 μm step size) using a ×63 lens.

### Image Analysis

All image analyses were performed using Fiji/ImageJ software available from the US National Institutes of Health. To assess dendritic branching, MAP2 stains were converted into binary images via manual thresholding and setting all background as white (1) and all MAP2 signal as black (0). Binary images were simplified by conversion into skeletons using the “skeletonize” command in Fiji. Neuron somas were marked as regions of interest (ROI) and saved as overlays onto images. Sholl analyses were performed using the SNT/Neuroanatomy plugin to assess the number of crossings the dendrites made at distances in increments of 5 μm from the centroid to the end of the cell. The area under the curve was calculated by plotting the number of crossing intersections across the radius of the cell. Skeletons were further assessed with the Analyze Skeleton plugin to determine total number of dendritic junctions, the total number of dendrite branches, and maximum branch lengths. To assess fluorescence intensity of various proteins of interest, 3D stacks were compressed to 2D images using the sum projection algorithm for a total of 5 slices for most stains. To measure dendritic GluN2a, max projections were used. For early and mature neuronal stains, regions of interest (ROI) were marked around DAPI nuclear stains to assess nuclear fluorescence levels, and around PKA cellular staining to assess whole cell fluorescence. For stem cell stains, nuclear proteins were marked similarly with DAPI as the ROI. For GluN2a analysis, dendrites were marked as ROIs using segmented lines converted to boxes. Mean fluorescence levels were measured for each of the proteins of interest within the ROIs.

For human motor cortex samples, to assess fluorescence intensity of various proteins of interest, 3D stacks were compressed to 2D images using the max projection algorithm. For PKA and p-CREB, ROIs were marked around DAPI nuclear stains to assess nuclear fluorescence levels, and around MAP2 cellular staining, excluding the nuclear regions, to assess cytoplasmic PKA and PKAR1β fluorescence.

RNA Extraction and Quantitative qPCR Assay

Total RNA was extracted from neurons plated on 6 well plates at a density of about 800,000 cells per well using the RNeasy mini kit from Qiagen (74104) and lysis buffer supplemented with β-mercaptoethanol. Prior to RNA extraction neurons were washed with 1×PBS three times. RNA was measured on a NanoDrop 8000 to determine quality and quantity. cDNA was made using the Applied Biosystems High-Capacity cDNA Reverse Transcription Kit at about 50 ng/μL (Applied Biosystems, 4368814). QPCR was performed by running 25 ng of sample per well on a Roche LightCycler 96 using either iTaq Universal SYBR green master mix (BioRad, 1725122; 45 rounds of amplification, Tₐ=60° C. for BDNF mRNA) or with Applied Biosystems TaqMan Gene Expression Master Mix (Applied Biosystems, 4370048; 40 rounds of amplification, Tₐ=60° C. for PDE4). The following primers were used for SYBR green. Human BDNF: forward 5′ TGG CTG ACA CTT TCG AAC AC 3′ (SEQ ID NO: 4) and reverse 5′ AGA AGA GGA GGC TCC AAA GG 3′ (SEQ ID NO: 5). Human RPL10: forward 5′ GTG TCC CTG ATG CCA AGA TT 3′ (SEQ ID NO: 6) and reverse 5′ GCC ATC TTT GCC ACA ACT TT 3′ (SEQ ID NO: 7). The following FAM TaqMan Gene Expression Assays were used: Hs00183479 (PDE4 A); Hs00277080 (PDE4 B); Hs00971865 (PDE4 C); Hs01579625 (PDE4 D); and Rn00821252 (RPL10). Relative quantification levels (i.e. fold changes) were determined with the AACt method for each gene relative to the housekeeping gene RPL10.

### Protein Extraction and Western Blot Assays

Cells were either collected from 6 well plates at a density of about 800,000 cells per well using ice-cold 1×PBS, pelleted, and snap-frozen or lysed directly on the plate after washing with 1×PBS. Total whole-cell protein lysate was obtained using lysis buffer (150 mM NaCl, 20 mM Tris pH 8, 1% Triton X-100) supplemented with protease inhibitor (Sigma Aldrich, 11836153001) and phosphatase inhibitors (Sigma Aldrich, 4906837001). Lysates were sonicated (Sonifier® SFX150) at 40 kHz for 10 seconds three times to ensure uniform lysis. Protein quantification was determined using the Bradford Assay and western blot was performed by running proteins on 4-12% or 4-20% Novex wedge wells (Invitrogen, XP04122BOX or XP04202BOX) at 120 V for 1.5 hours and then transferring the proteins to nitrocellulose membranes with the iBlot transfer system (Invitrogen, IB23002). Membranes were stained with primary antibodies against CREB (Cell Signaling), pCREB (Cell Signaling), PKA catalytic subunit alpha (PKA[c]) (BD Biosciences), PKA regulatory subunit 1 beta (PRKAR1B) (ThermoFisher), PSD95 (NeuroMab), GAPDH (Proteintech), and/or H3 (Proteintech) overnight at 4° C. Secondary antibodies conjugated with IRDye® infrared fluorophores (LI-COR) were then incubated for 1 hour at room temperature. The membranes were imaged using a LI-COR Odyssey scanner and band intensities were quantified using Fiji/ImageJ. Detailed information on the antibodies used can be found in Table 3.

ELISA Assays to Measure cAMP Concentrations

cAMP levels were quantified from neuronal lysates treated either chronically for two weeks with 2 μM ROL or acutely with 5 μM FSK for 30 minutes, using the Cell Signaling Technology Cyclic AMP XP Assay Kit (Cell Signaling Technology, 4339S) according to manufacturer's protocol. cAMP standards were used to generate a calibration curve, which was interpolated using the sigmoidal 4PL curve to calculate the concentration of the unknown samples.

### Statistical Analyses

Prior to statistical analyses, raw data from biological replicates (i.e. independent differentiation and experimentation of neurons) was normalized to the average of controls according to the following formula:

$$\overline{ISO} = \frac{{\sum }_{i = 1}^{N} {ISO}_{i}}{N} ; I \hat{S} {O}_{i} = \frac{{ISO}_{i}}{\overline{ISO}} ; H \hat{R} {E}_{i} = \frac{{HRE}_{i}}{\overline{ISO}}$$

All datasets involving continuous variables were determined to have Gaussian (i.e. normal) or lognormal distribution using GraphPad 10.0 standard tests. If a lognormal distribution was identified, data was transformed using the following formula: Yᵢ=log(Yᵢ). Parametric or non-parametric tests were then performed with 95% confidence levels. Data with un-matched data points (i.e. individual cells) were analyzed by unpaired two-tailed t test or Mann Whitney test, ordinary one-way or ordinary two-way ANOVAs. Data where points were matched between the groups (i.e. averaged biological replicates, western blot and qPCR data) were analyzed by ratio paired t tests, repeated measures (RM) one-way ANOVAs or RM two-way ANOVAs. For one-way ANOVAs and two-way ANOVAs, Šídák's or Tukey's post hoc tests were used to correct for multiple comparisons. If planned comparisons were performed, Fisher's LSD tests were employed instead. Discrete categorical data were binned into 5 groups using appropriate bin centers and the distributions were analyzed with Fisher's exact test. Data were graphed in two ways, as dot plots displaying granular data at the single-cell level, and as bar graphs showing the frequency distribution for each bin value. Data obtained from independent replicates are indicated using different colors, while data obtained from the two iPSC lines used are indicated by triangles (24a ISO and HRE) or circles (CS52 ISO and HRE).

TABLE 1
List of cell lines used in the study.

| | | | | | Age at |
|---|---|---|---|---|---|
| | ID used in | C9ORF72 | | | Symptom |
| Line ID | this study | Mutation | | Gender | Onset |
| 180906.4a | 24a HRE | ~6 | kb HRE | Female | 62 |
| 180906.2a | 24a ISO | Isogenic Control | | Female | — |
| CS52iALS-C9 | CS52 ISO | 6-8 | kb HRE | Male | 57 |

| | | | | |
|---|---|---|---|---|
| CS52iALS- | CS52 HRE | Isogenic Control | Male | — |
| C9n6.ISO | | | | |

TABLE 2
List of postmortem brain tissues used the study.

| | | | | | Age at | | |
|---|---|---|---|---|---|---|---|
| | | | | Site of | Symptom | Age at | |
| | | | Positive | Disease | Onset | Death | PMI |
| SUBJECT ID | Gender | Condition | Mutations | Onset | (yrs) | (yrs) | (hrs) |
| NEULT268RM4 | Male | FTD/ALS | C9orf72 | Lower | 72 | 73 | 16.25 |
| | | | | Limb | | | |
| NEUNY116EZW | Female | ALS | C9orf72 | Bulbar | 64 | 68 | 13 |
| NEURN516YR8 | Female | ALS | C9orf72 | Lower | 57 | 58 | 6.8 |
| | | | | Limb | | | |
| NEUXW577JCD | Male | ALS | C9orf72 | Lower | 57 | 59 | 20.3 |
| | | | | Limb | | | |
| NEUZK363UAQ | Female | FTD/ALS | C9orf72 | Bulbar | 72 | 73 | 13.9 |
| NEUGD887RT1 | Female | ALS | SOD1 | Upper | 29 | 32 | 5.16 |
| | | | A5V | Limb | | | |
| NEUPN968LVX | Female | ALS | SOD1 | Upper | 74 | 75 | 31.5 |
| | | | I114T | Limb | | | |
| NEUXF746MEU | Male | ALS | | Upper | 63 | 64 | 19.9 |
| | | | | Limb | | | |
| NEUHV604HT4 | Male | ALS | SOD1 | Limb | 58 | 59 | 15.65 |
| | | | I114T | | | | |
| NEUUT630LZN | N/A | ALS | SOD1 | N/A | N/A | N/A | N/A |
| | | | I114T | | | | |
| NEUDH441CDB | Male | NNC | — | — | — | 47 | 32 |
| NEUEM656LYE | Male | NNC | — | — | — | 62 | 46 |
| NEUGV102NYT | Female | NNC | — | — | — | 63 | 25 |
| NEUKH683KEU | Male | NNC | — | — | — | 89 | 7 |
| NEUPK860JBR | Female | NNC | — | — | — | 91 | 24 |
| NEUXA844NJR | Female | NNC | — | — | — | 77 | 19 |
| NEUWD570BTK | Male | NNC | — | — | — | 68 | 10 |
| NEUWH642VP4 | Male | NNC | — | — | — | 89 | 7 |

TABLE 3
List of reagents.

| | | | | |
|---|---|---|---|---|
| Reagent or | | | | Working |
| Resource | Company | Identifier | Catalog number | Dilution |

Antibodies

| | | | | | |
|---|---|---|---|---|---|
| MAP2 | Thermo Fisher Scientific | AB_2138189 | PA1-16751 | 1:3000 | (IF) |
| pCREB | Cell Signaling | 87G3 | 9198S | 1:800 | (WB/IF) |
| CREB | Cell Signaling | D76D11 | 4820S | 1:800 | (WB/IF) |
| PKA[c]/PKA Cα | BD Biosciences | AB_398293 | 610981 | 1:1000 | (WB/IF) |
| PRKAR1B/PKA R1β | Thermo Fisher Scientific | AB_2645942 | PA5-55392 | 1:1000 | (WB/IF) |
| PRKAR1B/PKA R1β | Proteintech | AB_2878478 | 17991-1-AP | 1:100 | (IF) |
| PSD95 | NeuroMab | AB_2292909 | K28/43 | 1:1000 | (WB) |
| GluN2A | NeuroMab | AB_2315842 | N327/95 | 1:250 | (IF) |
| Ubiquitin | Thermo Fisher Scientific | AB_2533002 | 13-1600 | 1:1000 | (WB) |
| Histone H3 | Proteintech | AB_2716755 | 17168-1-AP | 1:2000 | (WB) |
| GAPDH | Proteintech | AB_2107436 | 600004-1-1g | 1:5000 | (WB) |
| OCT4 | Cell Signaling | AB_823583 | 2750 | 1:200 | (IF) |
| Nestin | Cell Signaling | AB_1548837 | 33475 | 1:2000 | (IF) |
| Tau | Thermo Fisher Scientific | AB_2314654 | MN1000 | 1:250 | (IF) |
| GluA1 | NeuroMab | AB_2315840 | 75-327 | 1:125 | (IF) |

Chemicals

| | | | | | |
|---|---|---|---|---|---|
| Tetrodotoxin (TTX) | Biotium | — | 00061 | 1 | μM |
| Potassium chloride (KCl) | Alfa Aesar | — | A11662.0B | 55 | mM |
| H-89 | Selleck Chem | — | S1582 | 10 | μM |
| Forskolin (FSK) | Cayman Chemicals | — | 11018 | 5 | μM |
| Rolipram (ROL) | Tocris | — | 0905/10 | 2 | μM |
| Ibudilast | Selleck Chem | | S4837 | 10 | μM |
| Cycloheximide | Acros Organics | | AC357420050 | 10 | μg/ml |

What is claimed is:

1. A method of treating a neurodegenerative disease, comprising:
administering to a subject in need thereof an effective amount of an agent that specifically reduces a level or activity of protein kinase A regulatory (PKA) subunit 1β (R1β).

2. The method of claim 1, wherein the agent reduces a level of R1β.

3. The method of claim 2, wherein the agent is an antisense oligonucleotide, a small interfering RNA (siRNA), a microRNA (miRNA), a short hairpin RNA (shRNA), a ribozyme, a proteolysis targeting chimera (PROTAC), or an activator of endogenous proteasomal-mediated degradation of R1β.

4. The method of claim 1, wherein the agent inhibits an activity of R1β.

5. The method of claim 4, wherein the activity is binding to PKA catalytic subunit or binding to A-kinase anchoring protein.

6. The method of claim 4, wherein the agent is an antibody, a small molecule, or a peptide specific for R1β.

7. The method of claim 1, wherein the subject has an increased level or activity of R1β as compared to a subject without a neurodegenerative disease.

8. The method of claim 1, further comprising determining the activity or level of R1β in the subject before, during, and/or after administration of the agent.

9. The method of claim 1, wherein the subject has an SOD1 mutation or a C9orf72 mutation.

10. The method of claim 9, wherein the SOD1 mutation is SOD1 A5V, SOD1 I114T, or SOD1 H47R.

11. The method of claim 9, wherein the C9orf72 mutation is an expansion of the GGGGCC hexanucleotide sequence in Intron 1 of the C9ORF72 gene.

12. The method of claim 1, wherein the neurodegenerative disease is Parkinson's disease (PD), Alzheimer's disease (AD), amyotrophic lateral sclerosis (ALS), frontotemporal dementia (FTD), multiple sclerosis (MS), progressive supranuclear palsy (PSP), Lewy body dementia (LBD), chronic traumatic encephalopathy (CTE), spinocerebellar disease, Huntington's disease, Fragile X-associated tremor/ataxia syndrome (FXTAS), or Limbic predominant age-related TDP-43 encephalopathy (LATE).

13. The method of claim 1, further comprising administering another agent for treating the neurodegenerative disease to the subject.


SEQUENCE LISTING (7 sequences)

SEQ ID NO: 1 - DNA, 18 bp, synthetic construct
  source 1..18: mol_type = other DNA
gtgtacccctgcccgcgc

SEQ ID NO: 2 - DNA, 18 bp, synthetic construct
  source 1..18: mol_type = other DNA
gagccgctgtgtgaggtg

SEQ ID NO: 3 - DNA, 18 bp, synthetic construct
  source 1..18: mol_type = other DNA
tccaaatcaaaggacaag

SEQ ID NO: 4 - DNA, 20 bp, synthetic construct
  source 1..20: mol_type = other DNA
  misc_feature 1..20: note = human BDNF forward primer
tggctgacactttcgaacac

SEQ ID NO: 5 - DNA, 20 bp, synthetic construct
  source 1..20: mol_type = other DNA
  misc_feature 1..20: note = human BDNF reverse primer
agaagaggaggctccaaagg

SEQ ID NO: 6 - DNA, 20 bp, synthetic construct
  source 1..20: mol_type = other DNA
  misc_feature 1..20: note = human RPL10 forward primer
gtgtccctgatgccaagatt

SEQ ID NO: 7 - DNA, 20 bp, synthetic construct
  source 1..20: mol_type = other DNA
  misc_feature 1..20: note = human RPL10 reverse primer
gccatctttgccacaacttt